(19) United States
(12) Patent Application Publication
(10) Pub. No.: US 2026/0242487 A1
(43) Pub. Date: Aug. 20, 2026

(54) NOVEL ANTI-FGFR2 ANTIBODIES

(71) Applicant: HANGZHOU ZHONGMEIHUADONG PHARMACEUTICAL CO., LTD., Hangzhou (CN)

(72) Inventors: Tiantian ZHOU, Shanghai (CN); Qinglin DU, Shanghai (CN); Shuhua HAN, Shanghai (CN); Fei PENG, Shanghai (CN); Xueyan YANG, Shanghai (CN)

(21) Appl. No.: 18/852,417

(22) PCT Filed: Mar. 28, 2023
(86) PCT No.: PCT/CN2023/084213

§ 371 Date: Sep. 27, 2024

(30) Foreign Application Priority Data
Mar. 28, 2022 (WO) PCT/CN2022/083269

(51) Int. Cl. C07K 16/28; A61K 47/68; A61P 35/00

(52) U.S. Cl. CPC C07K 16/2863; A61K 47/6849; A61P 35/00; C07K 2317/41; C07K 2317/565; C07K 2317/732; C07K 2317/76; C07K 2317/92

(57) ABSTRACT

The present disclosure relates to an anti-FGFR2b antibody or antigen-binding fragments thereof, nucleic acid encoding the same, pharmaceutical compositions comprising the same, a method for producing the same, and the uses thereof.

### CROSS-REFERENCE TO RELATED APPLICATIONS

This application is a national stage of International Patent Application No. PCT/CN2023/084213, filed Mar. 28, 2023, which claims the benefit of CN Application No. PCT/CN2022/083269, filed Mar. 28, 2022, the contents of both of which are hereby incorporated by reference herein in their entireties.

### REFERENCE TO AN ELECTRONIC SEQUENCE LISTING

The contents of the electronic sequence listing (99SG-398586-US.xml; Size 18,697 bytes, and Date of Creation Sep. 27, 2024) is herein incorporated by reference in its entirety.

### FIELD OF THE INVENTION

The present disclosure relates to biomedicine field, and in particular to an antibody or antigen-binding fragment thereof that specifically binds to FGFR2b, a method for producing the same and the use thereof.

### BACKGROUND

Fibroblast growth factor receptors (FGFRs) are transmembrane tyrosine kinase receptors, and regulates many fundamental biological processes including embryogenesis, tissue and stem cell maintenance, angiogenesis, and wound healing through activation of the downstream PI3K-AKT and MAPK-ERK pathways (Beenken and Mohammadi, 2009; Katoh and Katoh, 2006; Turner and Grose, 2010). The FGFR family consists of 4 receptors (FGFR1 to FGFR4) corresponding to 22 ligands (FGF) (Korc and Friesel, 2009). Dysregulation of the FGFR2 signaling pathway leads to tumorigenesis and poor prognosis because of gene amplification, and resulting protein overexpression (Grose and Dickson, 2005). Splice variations result in several receptor variants, including 2 predominant FGFR2 isoforms, termed FGFR2b and FGFR2c (also named as FGFR2IIIb and FGFR2IIIc). The expression of each isoform is generally restricted to specific tissues. In particular, FGFR2b whose 3 major ligands are FGF7, FGF10, and FGF22 normally expressed on epithelial cells whereas FGFR2c expressed on mesenchymal tissues (Omitz et al., 1996; Zhang et al., 2006). In FGFR2-amplified gastric cancer, it is the FGFR2b isoform that is dominated.

In previous studies, FGFR2b is overexpressed in 2.5%-31.1% of Gastroesophageal adenocarcinoma (GEA) depending on the antibody and assay used (Ahn et al., 2016; Angal et al., 1993; Nagatsuma et al., 2015). Bemarituzumab (FPA144) is a humanized afucosylated immunoglobulin G1 monoclonal antibody specific to FGFR2b that blocking FGFR2b signaling by competitive binding inhibition of FGFs and eliciting enhanced antibody-dependent cell-mediated cytotoxicity (ADCC) against FGFR2b-overexpressing tumor cells (Xiang et al., 2021). Bemarituzumab demonstrated remarkable antitumor activity pre-clinically and positive efficacy in Phase I clinical trial (Catenacci et al., 2020; Xiang et al., 2021). However, there are three (10.7%) of 28 patients reported corneal toxicity receiving high dose (Catenacci et al., 2020). The mechanism of the corneal toxicity is hypothesized to be a result of inhibition of FGF10, which involved in the regulation of corneal epithelial wound healing (Itoh, 2016). In a previous in vivo study, the therapeutic efficacy of FGF10 was examined in an inflammation-induced rabbit dry eye model, indicating the effect of FGF10 in corneal epithelial cell healing (Zheng et al., 2015). There is a significant need for anti-FGFR2b antibodies to reduce corneal toxicity without affecting antitumor activity.

To develop a novel anti-FGFR2b antibody with low corneal toxicity to meet the unmet clinical needs, we generated a specific antibody against FGFR2b, which exhibited significant capacity of tumor suppression but low FGF10 inhibition implying low clinical adverse events.

### SUMMARY OF THE INVENTION

In one aspect, the present disclosure provides an antibody or antigen-binding fragment thereof which specifically binds to FGFR2b, comprises: a heavy chain complementarity determining region 1 (CDR1) having the amino acid sequence at least 80% identical to SEQ ID NO: 1, a heavy chain CDR2 having the amino acid sequence at least 80% identical to SEQ ID NO: 2, and a heavy chain CDR3 having the amino acid sequence at least 80% identical to SEQ ID NO: 3.

In some embodiments, the antibody or antigen-binding fragment thereof has at least one of the following properties:
i) weak inhibition on the binding of FGF10 to FGFR2b;
ii) indetectable binding affinity to FGFR2c.

In some embodiments, the antibody or antigen-binding fragment thereof comprises: a light chain CDR1 having the amino acid sequence of SEQ ID NO: 4; a light chain CDR2 having the amino acid sequence of SEQ ID NO: 5; and a light chain CDR3 having the amino acid sequence of SEQ ID NO: 6.

In some embodiments, the antibody or antigen-binding fragment thereof comprises: a heavy chain CDR1 consisting of the amino acid sequence set forth in SEQ ID NO: 1, a heavy chain CDR2 consisting of the amino acid sequence set forth in SEQ ID NO: 2, a heavy chain CDR3 consisting of the amino acid sequence set forth in SEQ ID NO: 3; and a light chain CDR1 consisting of the amino acid sequence set forth in SEQ ID NO: 4, a light chain CDR2 consisting of the amino acid sequence set forth in SEQ ID NO: 5, and a light chain CDR3 consisting of the amino acid sequence set forth in SEQ ID NO: 6.

In some embodiments, the antibody or antigen-binding fragment thereof comprises a heavy chain variable region having the amino acid sequence at least 80% identical to SEQ ID NO:7; and a light chain variable region having the amino acid sequence at least 80% identical to SEQ ID NO:8.

In some embodiments, the antibody or antigen-binding fragment thereof comprises a heavy chain variable region consisting of the amino acid sequence set forth in SEQ ID NO:7; and a light chain variable region consisting of the amino acid sequence set forth in SEQ ID NO:8.

In some embodiments, the antibody or antigen-binding fragment thereof comprises a heavy chain having the amino acid sequence of SEQ ID NO:9; and a light chain having the amino acid sequence of SEQ ID NO:10.

In some embodiments, the antibody or antigen-binding fragment thereof comprises an immunoglobulin constant region, optionally a constant region of human immunoglobulin, or optionally a constant region of human IgG. In a preferred embodiment, the constant region is derived from human IgG1.

In some embodiments, the constant region of the antibody or antigen-binding fragment thereof provided herein comprises one or more modifications which enhances antibody-dependent cellular cytotoxicity (ADCC).

In some embodiments, the antibody or antigen-binding fragment thereof provided herein is afucosylated.

In some embodiments, the antibody or antigen-binding fragment thereof provided herein is a human antibody, humanized antibody, chimeric antibody, monoclonal antibody, polyclonal antibody, recombinant antibody, diabody, triabody, tetrabody, Fab fragment, F(Fab′)₂ fragment, scFv fragment, Fv fragment, Fab′ fragment, or domain antibody.

In some embodiments, the antibody or antigen-binding fragment thereof provided herein is capable of inhibiting FGFR2 phosphorylation and proliferation of cancer cells induced by FGFs.

In another aspect, the present disclosure provides an antibody or antigen-binding fragment thereof which competes for binding to FGFR2b with the antibody or antigen-binding fragment thereof as described above.

In some embodiments, the antibody or antigen-binding fragment thereof specifically binds to the same epitope on FGFR2b recognized by an antibody or antigen-binding fragment thereof comprising heavy and light chain variable regions having the amino acid sequences set forth in SEQ ID NOs: 7 and 8, respectively.

In another aspect, the present disclosure provides a nucleic acid encoding the antibody or antigen-binding fragment thereof provided herein.

In some embodiments, the nucleic acid comprises: a heavy chain encoding nucleic acid having the nucleotide sequence of SEQ ID NO:11; and/or a light chain encoding nucleic acid having the nucleotide sequence of SEQ ID NO: 12.

In another aspect, the present disclosure provides an expression vector comprising the nucleic acid provided herein.

In another aspect, the present disclosure provides a host cell comprising the expression vector provided herein.

In another aspect, the present disclosure provides a pharmaceutical composition comprising:
(a) the antibody or antigen-binding fragment thereof provided herein, the nucleic acid provided herein, or the expression vector provided herein, and
(b) a pharmaceutically acceptable carrier.

In another aspect, the present disclosure provides an antibody-drug conjugate comprising the antibody or antigen-binding fragment thereof provided herein linked to one or more conjugate moieties.

In another aspect, the present disclosure provides a method of producing the antibody or antigen-binding fragment thereof provided herein, comprising culturing the host cell provided herein under conditions that allow the expression of the antibody or antigen-binding fragment thereof.

In another aspect, the present disclosure provides a use of the antibody or antigen-binding fragment thereof provided herein, the nucleic acid provided herein, the expression vector provided herein, the host cell provided herein, the pharmaceutical composition provided herein, or the antibody-drug conjugate provided herein, in the manufacture of a medicament for treating a disease or condition associated with the abnormal expression of FGFR2b in a subject.

In another aspect, the present disclosure provides a method of inhibiting or reducing FGF-induced proliferation of tumor cells in a subject, comprising administering a therapeutically effective amount of the antibody or antigen-binding fragment thereof provided herein, the nucleic acid provided herein, the expression vector provided herein, the host cell provided herein, or the pharmaceutical composition provided herein, or the antibody-drug conjugate provided herein to the subject.

In another aspect, the present disclosure provides a method of inhibiting or reducing FGF-induced FGFR2 phosphorylation in a subject, comprising administering a therapeutically effective amount of the antibody or antigen-binding fragment thereof provided herein, the nucleic acid provided herein, the expression vector provided herein, the host cell provided herein, the pharmaceutical composition provided herein, or the antibody-drug conjugate provided herein to the subject.

In another aspect, the present disclosure provides a method of killing tumor cells associated with the abnormal expression of FGFR2b and reducing corneal toxicity in a subject, comprising administering a therapeutically effective amount of the antibody or antigen-binding fragment thereof provided herein, the nucleic acid provided herein, the expression vector provided herein, the host cell provided herein, the pharmaceutical composition provided herein, or the antibody-drug conjugate provided herein to the subject.

In another aspect, the present disclosure provides a method of treating a disease or condition associated with the abnormal expression of FGFR2b in a subject, comprising administering a therapeutically effective amount of the antibody or antigen-binding fragment thereof provided herein, the nucleic acid provided herein, the expression vector provided herein, the host cell provided herein, the pharmaceutical composition provided herein, or the antibody-drug conjugate provided herein to the subject.

In some embodiments, the disease or condition as described above is a cancer, and optionally the cancer is characterized in expressing or over-expressing FGFR2b. In a preferred embodiment, the cancer is characterized in expressing or over-expressing FGFR2b.

In some embodiments, the disease or condition as described above is one selected from the group consisting of ovarian cancer, endometrial cancer, breast cancer, lung cancer, bladder cancer, colon cancer, prostate cancer, cervical cancer, colorectal cancer, pancreatic cancer, gastric cancer, esophageal cancer, hepatocellular carcinoma, renal cell carcinoma, head-and-neck cancer, mesothelioma, melanoma, sarcomas, brain tumors, gastroesophageal adenocarcinoma, malignant uterine neoplasm, adenocarcinoma of the gastroesophageal junction, cholangiocarcinoma, intrahepatic cholangiocarcinoma and urothelial caner. In a preferred embodiment, the disease or condition as described above is gastric cancer. In a preferred embodiment, the disease or condition as described above is FGFR2 positive gastric cancer. In a preferred embodiment, the disease or condition as described above is FGFR2-amplified gastric cancer.

In some embodiments, according to any one of the methods described above, the antibody or antigen-binding fragment thereof provided herein, the nucleic acid provided herein, the expression vector provided herein, the host cell provided herein, the pharmaceutical composition provided herein, or the antibody-drug conjugate provided herein is administered with at least one additional therapeutic agent sequentially or simultaneously.

In another aspect, the present disclosure provides a kit comprising the antibody or antigen-binding fragment thereof provided herein. In a preferred embodiment, the kit according to the present disclosure further comprises an instruction for guiding the use of the antibody or antigen-binding fragment thereof of the present disclosure, such as in treating or preventing a disease or condition associated with the abnormal expression of FGFR2b in a subject, such as a cancer.

These and other aspects are described in more detail herein. Each of the aspects provided may include a variety of embodiments provided herein. It is therefore expected that each of the aspects described may include each of the embodiments involving an element or a combination of elements, and all such combinations of the aspects and embodiments are explicitly taken into account.

### BRIEF DESCRIPTION OF THE DRAWINGS

FIG. 1A-B. Binding of anti-FGFR2b antibodies to different FGFR2 isoforms. FIG. 1A shows that HC18 specifically bound to hFGFR2b-expressing CHOK1 cells. FIG. 1B shows that HC18 did not bind to hFGFR2c-expressing CHOK1 cells.

FIG. 2A-B. Inhibition of anti-FGFR2b antibodies on the binding of FGFR2b to its ligands. FIG. 2A shows that HC18 significantly inhibited the binding of FGF7 to FGFR2b. FIG. 2B shows that HC18 partially inhibited the binding of FGF10 to FGFR2b.

FIG. 3. Surface plasmon response (SPR) sensorgrams of anti-FGFR2b antibodies binding to human FGFR2b antigen.

FIG. 4. ADCC (antibody dependent cellular cytotoxicity) bioassay using PBMC as effector cells induction with anti-FGFR2b antibodies and Ba/F3 cells expressing full-length human FGFR2b target cells (E:T=20:1).

FIG. 5A-B. Inhibition of FGFR2 phosphorylation induced by FGF7 (FIG. 5A) and FGF10 (FIG. 5B) of SNU-16 cells by anti-FGFR2b antibodies and negative control hIgG1.

FIG. 6A-B. Inhibition of ERK1/2 phosphorylation induced by FGF7 (FIG. 6A) and FGF10 (FIG. 6B) of SNU-16 cells by anti-FGFR2b antibodies and negative control hIgG1.

FIG. 7. Inhibition of the FGF7-induced proliferation of SNU-16 cells by anti-FGFR2b antibodies and negative control hIgG1.

### DETAILED DESCRIPTION OF THE INVENTION

Unless otherwise defined herein, the scientific and technical terms used herein should have the meaning generally understood by those skilled in the art. In addition, singular terms are intended to include plural terms and vice versa unless expressly indicated otherwise in the context. In order to facilitate reading the present application, certain terms are defined below.

### Definitions

As used herein, the indefinite articles “a” or “an” should be understood to refer to “one or more” of any recited or enumerated component.

As used herein, the term “about”, when applied to a numeric value, refers to a value that is reasonably close to the stated value and within an acceptable error range as determined by those skilled in the art, which will depend in part on how the value is measured or determined, i.e., the limitations of the measurement system. For example, “about” can mean a range of plus or minus 50% of a stated reference value, preferably a range of plus or minus 25%, or more preferably a range of plus or minus 10%. When particular values are provided in the application, the meaning of “about”, unless otherwise stated, should be understood to be within an acceptable error range for that particular value according to the practice in the art.

An “antibody” (Ab) shall include, without limitation, a glycoprotein immunoglobulin (Ig) which binds specifically to an antigen and comprises at least two heavy (H) chains and two light (L) chains interconnected by disulfide bonds, or an antigen-binding fragment thereof. Each H chain comprises a heavy chain variable region (abbreviated herein as V_{H}) and a heavy chain constant region. The heavy chain constant region of an IgG Ab comprises three constant domains, CH₁, CH₂ and CH₃. Each light chain comprises a light chain variable region (abbreviated herein as V_{L}) and a light chain constant region. The light chain constant region of an IgG Ab comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs (light chain CDRs including LCDR1, LCDR2, and LCDR3, heavy chain CDRs including HCDR1, HCDR2, HCDR3) and four FRs, arranged from amino-terminus to carboxy-terminus in the following order: FR1-CDR1-FR2-CDR2-FR3-CDR3-FR4. The variable regions of the heavy and light chains contain a binding domain that interacts with an antigen. A variety of methods have been used to delineate the CDR domains within an Ab, including the Rabat, Chothia, AbM, contact, and IMGT definitions. Unless specifically indicated, Kabat numbering is used in the present disclosure as a default. The constant regions of the Abs may mediate the binding of the Ig to host tissues or factors, including various cells of the immune system (e.g., effector cells) and the first component (Clq) of the classical complement system. Antibodies are assigned to classes based on the amino acid sequence of the constant region of their heavy chain. The five major classes or isotypes of antibodies are IgA, IgD, IgE, IgG, and IgM. Several of the major antibody classes are divided into subclasses such as IgG1, IgG2, IgG3, IgG4, IgAQ1, or IgA2.

As used herein, the term “antibody” includes any immunoglobulin, monoclonal antibody, polyclonal antibody, multivalent antibody, bivalent antibody, monovalent antibody, multispecific antibody, bispecific antibody that binds to a specific antigen. An intact antibody or an antibody fragment having the antigen binding portion of the antibody can be used. The term “antigen-binding fragment” as used herein refers to an antibody fragment formed from a portion of an intact antibody comprising one or more CDRs, or any other antibody fragment that can bind to an antigen but does not comprise an intact native antibody structure. The term “antibody or antigen-binding fragment thereof” as used herein refers to an intact antibody or an antibody fragment having an antigen-binding portion. The antigen-binding portion can be produced by recombinant DNA techniques or by enzymatic or chemical cleavage of an intact antibody. The antibody or antigen-binding fragment thereof includes, without limitation, Fab, Fab′, F(ab′)₂, Fv, domain antibody (dAb), fragments comprising complementarity determining regions (CDRs), single chain variable fragment (scFv), chimeric antibody, diabody, triabody, tetrabody, and a polypeptide containing at least a portion of the immunoglobulin that is sufficient to impart specific antigen-binding to the polypeptide.

“Fab fragment” is a monovalent fragment having the V_{L}, V_{H}, C_{L}, and CHI domains. “F(ab′)₂ fragment” is a bivalent fragment having two Fab fragments connected in the hinge region by a disulfide bond. “Fv fragment” has the V_{L} and V_{H} domains derived from a single arm of an antibody. “domain antibody (dAb)” consists of the V_{H} or V_{L} domains. “Single-chain variable fragment (scFv)” is an antibody in which the V_{L} and V_{H} regions are connected to form a continuous protein chain via a linker (e.g., a synthetic sequence of amino acid residues), wherein the linker is sufficient in length to allow the protein chain to form a monovalent antigen binding site. The term “diabody” is a divalent antibody comprising two polypeptide chains, wherein each polypeptide chain comprises the V_{H} and V_{L} domains connected by a linker that is too short to allow the two domains on the same chain to be paired, thereby allowing each domain to be paired with the complementary domain on the other polypeptide chain. If the two polypeptide chains of the diabody are same, the resulted diabody will have two identical antigen binding sites. The polypeptide chains having different sequences can be used to produce a diabody or a bispecific antibody having two different antigen binding sites. The diabody or bispecific antibody also refers to an artificial antibody or an antigen-binding fragment which has fragments derived from two different monoclonal antibodies and is capable of binding to two different epitopes. The two epitopes may present on the same antigen, or they may present on two different antigens. Similarly, the triabody, tetrabody or other multispecific antibody are antibodies comprising three, four or multiple polypeptide chains that may be same or different, and thus form three, four or multiple antigen binding sites that may be same or different, respectively.

As used herein, the term “human” Ab refers to an Ab having variable regions in which both the framework and CDR regions are derived from human germline immunoglobulin sequences. Furthermore, if the Ab contains a constant region, the constant region also is derived from human germline immunoglobulin sequences.

As used herein, the term “humanized” Ab refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Minor additions, deletions, insertions, substitutions or modifications of amino acids are permissible as long as they do not abrogate the ability of the Ab to bind to a particular antigen. A “humanized” Ab retains an antigenic specificity similar to that of the original Ab.

As used herein, the term “monoclonal” Ab (mAb) refers to a non-naturally occurring preparation of Ab molecules of single molecular composition, i.e., Ab molecules whose primary sequences are essentially identical and which exhibit a single binding specificity and affinity for a particular epitope. MAbs may be produced by hybridoma, recombinant, transgenic or other techniques known to those skilled in the art.

As used herein, the term “chimeric” Ab refers to an Ab in which the variable regions are derived from one species and the constant regions are derived from another species, such as an Ab in which the variable regions are derived from a mouse Ab and the constant regions are derived from a human Ab. In an illustrative example, a chimeric antibody may comprise a constant region derived from human and a variable region from a non-human animal such as mouse. In some embodiments, the non-human animal is a mammal, for example, a mouse, a rat, a rabbit, a goat, a sheep, a guinea pig, or a hamster.

As used herein, the term “specific binding” or “specifically binds” refers to a non-random binding reaction between two molecules, such as for example between an antibody and an antigen. Binding affinity of the antibody and antigen-binding fragment provided herein can be represented by K_{D} value, which represents the ratio of dissociation rate to association rate (koff/kon) when the binding between the antigen and antigen-binding molecule (e.g. the antibody and antigen-binding fragment) reaches equilibrium. The antigen-binding affinity (e.g. K_{D}) can be appropriately determined using suitable methods known in the art, including, for example, Biacore techniques, Kinexa techniques, and flow cytometry.

As used herein, the term “compete for binding” refers to the ability of an antibody or antigen-binding fragment to inhibit the binding interaction between two molecules (e.g. human FGFR2b and an anti-FGFR2b antibody) to any detectable degree (e.g. by at least 85%, or at least 90%, or at least 95%). Those skilled in the art will recognize that it is possible to determine, without undue experimentation, if a given antibody competes for binding to FGFR 2b with the antibody of present disclosure.

As used herein, the term “epitope” refers to the specific group of atoms or amino acids on an antigen to which an antibody or an antigen-binding portion binds. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure. The CDRs are important for recognizing an epitope of an antigen.

As used herein, “percent (%) identical to” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences. Sequence identity refers to exact matches between the nucleotides or amino acids of two sequences which are being compared. Sequence identity can be determined by those skilled in the art through conventional means, such as BLAST algorithm.

As used herein, “Antibody-dependent cell-mediated cytotoxicity” (“ADCC”) refers to an in vitro or in vivo cell-mediated cytotoxic activity in which nonspecific effector cells that express Fc receptors (FcRs) on the effector cell surface (e.g., natural killer (NK) cells, macrophages, neutrophils and eosinophils) recognize the Fc region of the Abs bound to surface antigens on a target cell and actively lyses the target cell. In principle, any effector cell with an activating FcR can be triggered to mediate ADCC. ADCC activity of an Ab can be measured as described in Example 5, or by any methods known to those skilled in the art.

As used herein, a modified Ab comprises one or more modifications that “enhances ADCC” means the ADCC activity level of the modified Ab is greater than ADCC induced by an unmodified Ab. For example, the enhanced ADCC as described in the present disclosure is characterized in at least about 10%, about 15%, about 20%, about 25%, about 30%, about 35%, about 40%, about 45%, about 50%, about 60%, about 65%, about 70%, or about 75% higher lysis of FGFR2b expressing cell. Various methods for ADCC enhancement have been described in prior art (Liu R, Oldham R J, Teal E, Beers S A, Cragg M S. Antibodies (Basel). 2020 Nov. 17; 9(4):64). For example, studies have shown that afucosylated (i.e., fucose deficient, or non-fucosylated) antibody exhibited an increased binding to FcγRIII and thus provoked a higher ADCC activity (Shields et al. (2002) J. Biol. Chem., 277: 26733-26740; Shinkawa et al. (2003) J. Biol. Chem., 278: 3466-3473; and European Patent Appln. Pub. No. 1176195). In some embodiments, the afucosylated antibody provided herein lacks fucose at asparagine 297 (Asn297) of the heavy chain. Asn297 (Eu numbering of Fc region residues; or position 314 in Kabat numbering) is a conserved N-linked glycosylation site found in each CH₂ domain of the Fc region of IgG1 isotype of antibodies.

As used herein, “administering”, “administered” or “administration” refers to the physical introduction of a composition comprising a therapeutic agent to a subject, using any of the various methods and delivery systems known to those skilled in the art. A preferred route for administration of therapeutic antibodies is intravenous (IV) administration. Other routes of administration include subcutaneous (SC), intraperitoneal (IP), intramuscular (IM), spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, the antibody or antigen-binding fragment thereof according to the present disclosure can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “fibroblast growth factor receptor 2 (FGFR2)” also known as CD332 (cluster of differentiation 332) is one member of the FGFR family that in humans encoded by the FGFR2 gene residing on chromosome 10. FGFRs are highly conserved, having common structural features which consist of an extracellular ligand-binding section composed of different Ig-like domains (a isoform contains all three Ig-like domains D1, D2, and D3; β isoform contains only the two Ig-like domains D2 and D3 domains but without D1), a transmembrane domain, and an intracellular tyrosine kinase catalytic domain. FGFR2 has two naturally occurring isoforms, FGFR2IIIb and FGFR2IIIc, created by splicing of the third immunoglobulin-like domain (D3). FGFR2IIIb is a high affinity receptor for FGF1 and is the specific receptor for the KGF family members (e.g., FGF 10, FGF22, and especially FGF7). KGF (FGF7) and KGFR (FGFR2IIIb) are found abnormally expressed in various types of cancers such as pancreatic cancer, gastric cancer, ovarian cancer and breast cancer (Helsten T, Elkin S, Arthur E, Tomson B N, Carter J, Kurzrock R. Clin Cancer Res. 2016 Jan. 1; 22(1):259-67).

“Abnormal expression of FGFR2b” as used herein, includes, without limitation, FGFR2b mutation, FGFR2b amplification, FGFR2b fusion, FGFR2 translocation, and FGFR2 overexpression.

As used herein, “anti-FGFR2b antibodies” refers to antibodies that can specifically bind to human or non-human FGFR2b (e.g., proteins disclosed as UniProtKB-P21802-3, UniProtKB-AOA2K5TL84, UniProtKB-P21803-2 in UniProt database). In some embodiment, the anti-FGFR2b antibodies can specifically bind to FGFR2b with a binding affinity (K_{D}) less than 1×10⁻⁴ M, less than 1×10⁻⁵ M, less than 1×10⁻⁶ M, less than 1×10⁻⁷ M, less than 1×10⁻⁸ M, less than 1×10⁻⁹ M, or less than 1×10⁻¹⁰ M. In some embodiments, the anti-FGFR2b antibodies can specifically bind to FGFR2b with a K_{D} less than 50 nM, 30 nM, 20 nM, 15 nM, 10 nM, 9 nM, 8 nM, 7 nM, 6 nM, 5 nM, 4 nM, 3 nM, 2 nM, or 1 nM. In a preferred embodiment, the anti-FGFR2b antibodies specifically bind to FGFR2b with a binding affinity (K_{D}) less than 1×10⁻⁷ M, less than 1×10⁻⁸ M, less than 1×10⁻⁹ M, or less than 1×10⁻¹⁰ M.

As used herein, the term “cancer” refers to a broad group of various diseases characterized by the uncontrolled growth of abnormal cells in the body. Unregulated cell division and growth result in the formation of malignant tumors that invade neighboring tissues and may also metastasize to distant parts of the body through the lymphatic system or bloodstream. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the antibody or antigen-binding fragment thereof provided herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

As used herein, “subject” includes any human or non-human animal. The term “non-human animal” includes, without limitation, vertebrates such as non-human primates, sheep, dogs, monkey, chimpanzee, gorilla, and rodents such as mice, rats and guinea pigs. In preferred embodiments, the subject is a human. The terms “subject” and “patient” are used interchangeably herein.

As used herein a “vector” refers to a polynucleotide molecule which enables replicating/cloning of a desired nucleic acid fragment contained therein, or enables expressing of a protein encoded by such desired nucleic acid fragment as introduced into an appropriate cell host. Examples of vectors include both cloning vectors and expression vectors. The term “expression vector” as used herein refers to a vehicle into which a polynucleotide encoding a protein may be operably inserted so as to bring about the expression of that protein. An expression vector may contain a variety of elements for controlling expression, including promoter sequences, transcription initiation sequences, enhancer sequences, selectable elements, and reporter genes. In addition, the vector may contain an origin of replication. A vector can be introduced into the host cell by methods known in the art, e.g., electroporation, chemical transfection (e.g., DEAE-dextran), transformation, transfection, and infection and/or transduction (e.g., with recombinant virus). Non-limiting examples of vectors include viral vectors (which can be used to generate recombinant virus), naked DNA or RNA, plasmids, cosmids, phage vectors, and DNA or RNA expression vectors associated with cationic condensing agents.

As used herein, the “host cell” means a cell that has been transformed or is capable of being transformed with a nucleic acid sequence and thus expresses a target gene. The host cell can be prokaryotic (e.g., E. coli), eukaryotic (e.g., yeast, plant including tobacco and tomato, animals including human, monkey, hamster, rat, mouse, or insect), or hybridomas.

As used herein, “therapeutically effective amount” or “therapeutically effective dosage” of a drug or therapeutic agent is any amount of the drug or agent that, when used alone or in combination with another therapeutic agent, protects a subject against the onset of a disease or promotes disease regression evidenced by a decrease in severity of disease symptoms, an increase in frequency and duration of disease symptom-free periods, a prevention or reduction of impairment or disability due to the disease affliction, or otherwise an amelioration of disease symptoms in the subject. In addition, the terms “effective” and “effectiveness” with regard to a treatment includes both pharmacological effectiveness and physiological safety. Pharmacological effectiveness refers to the ability of the drug to promote disease regression, e.g., cancer regression, in the patient. Physiological safety refers to an acceptable level of toxicity, or other adverse physiological effects at the cellular, organ and/or organism level (adverse effects) resulting from administration of the drug. The efficacy of a therapeutic agent can be evaluated using a variety of methods known to the practitioner, such as in human subjects during clinical trials, in animal model systems predictive of efficacy in humans, or by assaying the activity of the agent in in vitro assays.

As used herein, the term “potent inhibition” refers to the antibody or antigen-binding fragment thereof of the present disclosure has a relative inhibition rate of at least about 75%, at least about 80%, at least about 85%, at least about 90%, at least about 95%, at least about 100%, as compared with reference antibody. The term “weak inhibition” refers to the antibody or antigen-binding fragment thereof of the present disclosure has a relative inhibition rate of at most about 65%, at most about 60%, at most about 55%, at most about 50%, at most about 45%, at most about 40%, as compared with reference antibody. In some embodiments, the anti-FGFR2b reference antibody is FPA144. In some preferred embodiments, the term “potent inhibition” refers to the antibody or antigen-binding fragment thereof of the present disclosure has a relative inhibition rate of at least about 85%, at least about 90%, at least about 95%, at least about 100%, as compared with reference antibody FPA144. In some preferred embodiments, the term “weak inhibition” refers to the antibody or antigen-binding fragment thereof of the present disclosure has a relative inhibition rate of at most about 55%, at most about 50%, at most about 45%, at most about 40%, as compared with reference antibody FPA144.

As used herein, the term “treating” or “treatment” of a disease or condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, the term “pharmaceutically acceptable carrier” indicates that the designated carrier, vehicle, diluent, excipient(s), and/or salt is generally chemically and/or physically compatible with the other ingredients comprising the formulation, and physiologically compatible with the recipient thereof.

### Anti-FGFR2b Antibodies and Antigen-Binding Fragments

The present disclosure provides a novel anti-FGFR2b antibody (e.g., HC18) or antigen-binding fragment thereof, specifically binds to FGFR2b, such as human FGFR2b expressed the surface of a cell, but does not have detectable binding affinity to FGFR2c. In addition, the antibody or antigen-binding fragment thereof of the present disclosure mediates potent inhibition on the binding of FGF7 to FGFR2b but weak inhibition on the binding of FGF10 to FGFR2b, which provides an improved balance of safety/efficacy.

The experiments showed that the antibody provided herein displayed significant blocking of FGF7-FGFR2b pathway by inhibition of FGF7-induced FGFR2b and ERK1/2 phosphorylation and tumor cell proliferation (e.g., SNU-16 cells), and showed relatively weak blocking of FGF10 binding to FGFR2b compared with benchmark (e.g., FPA144), suggesting that it may have better safety profile in corneal toxicity. An ADCC function assay revealed that the antibody of the present disclosure induced strong ADCC effects to kill FGFR2b-expressing tumor cells.

The antibody or antigen-binding fragment thereof provided herein comprises: a heavy chain complementarity determining region 1 (CDR1) having the amino acid sequence at least 80% (e.g., at least 85%, 90%, 95%, 96%, 97%, 98%, 99% or 100%) identical to SEQ ID NO: 1 or having up to 1 amino acid addition, substitution and/or deletion compared with SEQ ID NO: 1, a heavy chain CDR2 having the amino acid sequence at least 80% (e.g., at least 85%, 90%, 95%, 96%, 97%, 98%, 99% or 100%) identical to SEQ ID NO: 2 or having up to 3 (e.g., 1, 2 or 3) amino acid additions, substitutions and/or deletions compared with SEQ ID NO: 2, and a heavy chain CDR3 having the amino acid sequence at least 80% (e.g., at least 85%, 90%, 95%, 96%, 97%, 98%, 99% or 100%) identical to SEQ ID NO: 3 or having up to 1 amino acid addition, substitution and/or deletion compared with SEQ ID NO: 3.

In some embodiments, the antibody or antigen-binding fragment thereof comprises: a light chain CDR1 having the amino acid sequence at least 80% (e.g., at least 85%, 90%, 95%, 96%, 97%, 98%, 99% or 100%) identical to SEQ ID NO: 4 or having up to 2 (e.g., 1 or 2) amino acid additions, substitutions and/or deletions compared with SEQ ID NO: 4; a light chain CDR2 having the amino acid sequence at least 80% (e.g., at least 85%, 90%, 95%, 96%, 97%, 98%, 99% or 100%) identical to SEQ ID NO: 5 or having up to 1 amino acid addition, substitution and/or deletion compared with SEQ ID NO:5; and a light chain CDR3 having the amino acid sequence at least 80% (e.g., at least 85%, 90%, 95%, 96%, 97%, 98%, 99% or 100%) identical to SEQ ID NO: 6 or having up to 1 amino acid addition, substitution and/or deletion compared with SEQ ID NO: 6.

In a preferred embodiment, the antibody or antigen-binding fragment thereof comprises: a heavy chain CDR1 having the amino acid sequence of SEQ ID NO: 1; a heavy chain CDR2 having the amino acid sequence of SEQ ID NO: 2; and a heavy chain CDR3 having the amino acid sequence of SEQ ID NO: 3.

In a preferred embodiment, the antibody or antigen-binding fragment thereof comprises: a light chain CDR1 having the amino acid sequence of SEQ ID NO: 4; a light chain CDR2 having the amino acid sequence of SEQ ID NO: 5; and a light chain CDR3 having the amino acid sequence of SEQ ID NO: 6.

In a preferred embodiment, the antibody or antigen-binding fragment thereof comprises the CDR sequences of HC18 (see Table 1).

TABLE 1
The CDR Sequences of HC18.

| | | | |
|---|---|---|---|
| HC18 | HCDR1 | HCDR2 | HCDR3 |
| | SEQ ID NO: 1 | SEQ ID NO: 2 | SEQ ID NO: 3 |
| | SYWMH | SIYPENSDTSYNQKFKG | YHGYDGDY |
| | LCDR1 | LCDR2 | LCDR3 |
| | SEQ ID NO: 4 | SEQ ID NO: 5 | SEQ ID NO: 6 |
| | SASSSVSYMY | STSNLAS | QQRSSYPYT |

In a preferred embodiment, the antibody or antigen-binding fragment thereof comprises: a heavy chain CDR1 consisting of the amino acid sequence set forth in SEQ ID NO: 1, a heavy chain CDR2 consisting of the amino acid sequence set forth in SEQ ID NO: 2, a heavy chain CDR3 consisting of the amino acid sequence set forth in SEQ ID NO: 3; a light chain CDR1 consisting of the amino acid sequence set forth in SEQ ID NO: 4, a light chain CDR2 consisting of the amino acid sequence set forth in SEQ ID NO: 5, and a light chain CDR3 consisting of the amino acid sequence set forth in SEQ ID NO: 6.

CDRs are known to be responsible for antigen binding, however, it has been found that not all of the six CDRs are indispensable or unchangeable. In other words, it is possible to replace or change or modify one or more CDRs in HC18, yet substantially retain the specific binding affinity to FGFR2b.

In some embodiments, the antibody or antigen-binding fragment thereof provided herein may comprise one or more modifications or substitutions in one or more CDR regions as provided in Table 1. Such variants retain specific binding affinity to FGFR2b of their parent antibody, but may have one or more improvement in properties such as higher antigen-binding affinity or reduced likelihood of glycosylation.

In some embodiments, the antibody or antigen-binding fragment thereof provided herein may be modified to remove one or more Asn or Asp hotspots within the CDR regions (or within the variable regions). Such Asn and Asp hotspots may lead to degradation of the antibodies and consequently reduce the stability of the antibodies.

In some embodiment, the one or more modification or substitution is a conservative substitution.

The antibody or antigen-binding fragment thereof provided herein further comprises suitable framework region (FR) sequences, as long as the antibodies can specifically bind to FGFR2b. The CDR sequences provided in Table 1 are obtained from a mouse antibody, but they can be grafted to any suitable FR sequences of any suitable species such as mouse, human, rat, rabbit, among others, using suitable methods known in the art such as recombinant techniques. In some embodiments, the antibody or antigen-binding fragment thereof provided herein are humanized.

In some embodiments, the antibody or antigen-binding fragment thereof comprises a heavy chain variable region having the amino acid sequence at least 80% (e.g., at least 85%, 90%, 95%, 96%, 97%, 98%, 99% or 100%) identical to SEQ ID NO:7 or having up to 20 (e.g., 1, 2, 3, 4, 5, 6, 7, 8, 9, 10, 11, 12, 13, 14, 15, 16, 17, 18, 19, or 20) amino acid additions, substitutions and/or deletions compared with SEQ ID NO:7 (i.e., the heavy chain variable regions for HC18); and a light chain variable region having the amino acid sequence at least 80% (e.g., at least 85%, 90%, 95%, 96%, 97%, 98%, 99% or 100%) identical to SEQ ID NO:8 or having up to 20 (e.g., 1, 2, 3, 4, 5, 6, 7, 8, 9, 10, 11, 12, 13, 14, 15, 16, 17, 18, 19, or 20) amino acid additions, substitutions and/or deletions compared with SEQ ID NO:8 (i.e., the light chain variable regions for HC18). In a preferred embodiment, the substitution is a conservative substitution.

In a preferred embodiment, the antibody or antigen-binding fragment thereof comprises a heavy chain variable region consisting of the amino acid sequence set forth in SEQ ID NO:7; and a light chain variable region consisting of the amino acid sequence set forth in SEQ ID NO:8.

The antibody or antigen-binding fragment thereof provided herein further comprises an immunoglobulin constant region, optionally a constant region of human immunoglobulin, optionally a constant region of human IgG. In some embodiments, an immunoglobulin constant region comprises a heavy chain and/or a light chain constant region. The heavy chain constant region comprises CH₁, hinge, and/or CH₂-CH₃ regions. In some embodiments, the heavy chain constant region comprises a Fc region. In some embodiments, the light chain constant region comprises C_{κ} or C_{λ}. In a preferred embodiment, the constant region is derived from human IgG1(hIgG1). In a preferred embodiment, the constant region is a constant region of human IgG1.

In some embodiments, the antibody or antigen-binding fragment thereof provided herein comprises at least one heavy chain and/or at least one light chain. In an embodiment, the heavy chain having the amino acid sequence at least 85% (e.g., at least 90%, 95%, 96%, 97%, 98%, 99% or 100%) identical to SEQ ID NO:9 or having up to 50 (e.g., 1, 2, 3, 4, 5, 6, 7, 8, 9, 10, 15, 20, 25, 30, 35, 40, 45 or 50) amino acid additions, substitutions and/or deletions compared with SEQ ID NO:9 (i.e., the full-length heavy chain sequence of HC18). In an embodiment, the light chain having the amino acid sequence at least 85% (e.g., at least 90%, 95%, 96%, 97%, 98%, 99% or 100%) identical to SEQ ID NO: 10 or having up to 50 (e.g., 1, 2, 3, 4, 5, 6, 7, 8, 9, 10, 15, 20, 25, 30, 35, 40, 45 or 50) amino acid additions, substitutions and/or deletions compared with SEQ ID NO: 10 (i.e., the full-length light chain sequence of HC18). In a preferred embodiment, the substitution is a conservative substitution.

In a preferred embodiment, the antibody or antigen-binding fragment thereof comprises a heavy chain having the amino acid sequence of SEQ ID NO:9; and a light chain having the amino acid sequence of SEQ ID NO: 10.

Table 2 shows the amino acid sequences and nucleotide sequences of HC18.

TABLE 2
The amino acid sequences and nucleotide sequences of HC18.

| HC18 | Sequences |
|---|---|
| V_{H} (SEQ ID | EVQLQQSGTVLARPGASVKMSCKTSGYTFTSYWMHWVKQRPG |
| No: 7) | QGLEWIGSIYPENSDTSYNQKFKGKAKLTAVTSASTAYMDLSSLT |
| | NEDSAVYYCSIYHGYDGDYWGQGTTLTVSS |
| V_{L} (SEQ ID | QIVLTQSPAIMSASPGEKVTITCSASSSVSYMYWFQQKPGTSPKL |
| No: 8) | WIYSTSNLASGVPARFSGSGSGTSYSLTISRMEAEDAATYYCQQR |
| | SSYPYTFGGGTKLEIK |
| Full-length | EVQLQQSGTVLARPGASVKMSCKTSGYTFTSYWMHWVKQRPG |
| heavy chain | QGLEWIGSIYPENSDTSYNQKFKGKAKLTAVTSASTAYMDLSSLT |
| (SEQ ID | NEDSAVYYCSIYHGYDGDYWGQGTTLTVSSASTKGPSVFPLAPS |
| No: 9) | SKSTSGGTAALGCLVKDYFPEPVTVSWNSGALTSGVHTFPAVLQ |
| | SSGLYSLSSVVTVPSSSLGTQTYICNVNHKPSNTKVDKKVEPKSC |
| | DKTHTCPPCPAPELLGGPSVFLFPPKPKDTLMISRTPEVTCVVVD |
| | VSHEDPEVKFNWYVDGVEVHNAKTKPREEQYNSTYRVVSVLT |
| | VLHQDWLNGKEYKCKVSNKALPAPIEKTISKAKGQPREPQVYT |
| | LPPSRDELTKNQVSLTCLVKGFYPSDIAVEWESNGQPENNYKTTP |
| | PVLDSDGSFFLYSKLTVDKSRWQQGNVFSCSVMHEALHNHYTQ |
| | KSLSLSPGK |
| Full-length | QIVLTQSPAIMSASPGEKVTITCSASSSVSYMYWFQQKPGTSPKL |
| light chain | WIYSTSNLASGVPARFSGSGSGTSYSLTISRMEAEDAATYYCQQR |
| (SEQ ID | SSYPYTFGGGTKLEIKRTVAAPSVFIFPPSDEQLKSGTASVVCLLN |
| No: 10) | NFYPREAKVQWKVDNALQSGNSQESVTEQDSKDSTYSLSSTLT |
| | LSKADYEKHKVYACEVTHQGLSSPVTKSFNRGEC |
| Nucleotide | GAGGTGCAGCTGCAGCAGAGCGGGACAGTGCTGGCCAGACC |
| sequence | AGGAGCAAGCGTGAAGATGAGCTGTAAGACAAGCGGGTACA |
| encoding | CATTTACAAGCTATTGGATGCACTGGGTGAAGCAGAGACCCG |
| heavy chain | GCCAAGGCCTGGAGTGGATCGGGAGCATCTACCCCGAGAACA |
| full-length | GCGATACAAGCTACAACCAGAAATTCAAGGGCAAGGCCAAGC |
| sequence | TGACCGCCGTGACCAGCGCCAGCACCGCATACATGGACCTGA |
| (SEQ ID | GCAGCCTGACCAACGAGGACAGCGCAGTGTACTACTGCAGCA |
| No: 11) | TCTACCACGGCTACGACGGAGACTACTGGGGCCAAGGCACCA |
| | CTCTCACCGTGAGCAGCGCTAGCACCAAGGGCCCATCGGTCT |
| | TCCCCCTGGCACCCTCCTCCAAGAGCACCTCTGGGGGCACAG |
| | CGGCCCTGGGCTGCCTGGTCAAGGACTACTTCCCCGAACCGG |
| | TGACGGTGTCGTGGAACTCAGGCGCCCTGACCAGCGGCGTGC |
| | ACACCTTCCCGGCTGTCCTACAGTCCTCAGGACTCTACTCCCT |
| | CAGCAGCGTGGTGACCGTGCCCTCCAGCAGCTTGGGCACCCA |
| | GACCTACATCTGCAACGTGAATCACAAGCCCAGCAACACCAA |
| | GGTGGACAAGAAAGTTGAGCCCAAATCTTGTGACAAAACTCA |
| | CACATGCCCACCGTGCCCAGCACCTGAACTCCTGGGGGGACC |
| | GTCAGTCTTCCTCTTCCCCCCAAAACCCAAGGACACCCTCATG |
| | ATCTCCCGGACCCCCGAGGTCACATGCGTGGTGGTGGACGTG |
| | AGCCACGAAGACCCTGAGGTCAAGTTCAACTGGTACGTGGAC |
| | GGCGTGGAGGTGCATAATGCCAAGACAAAGCCGCGGGAGGA |
| | GCAGTACAACAGCACGTACCGTGTGGTCAGCGTCCTCACCGT |
| | CCTGCACCAGGACTGGCTGAATGGCAAGGAGTACAAGTGCAA |
| | GGTCTCCAACAAAGCCCTCCCAGCCCCCATCGAGAAAACCAT |
| | CTCCAAAGCCAAAGGGCAGCCCCGAGAACCACAGGTGTACA |
| | CCCTGCCCCCATCCCGGGACGAGCTGACCAAGAACCAGGTCA |
| | GCCTGACCTGCCTGGTCAAAGGCTTCTATCCCAGCGACATCGC |
| | CGTGGAGTGGGAGAGCAATGGGCAGCCGGAGAACAACTACA |
| | AGACCACGCCTCCCGTGCTGGACTCCGACGGCTCCTTCTTCCT |
| | CTACAGCAAGCTCACCGTGGACAAGAGCAGGTGGCAGCAGG |
| | GGAACGTCTTCTCATGCTCCGTGATGCATGAGGCTCTGCACAA |
| | CCACTACACGCAGAAGAGCCTCTCCCTGTCTCCGGGTAAATGA |
| | TTCTAGA |
| Nucleotide | CAGATCGTCCTCACCCAAAGCCCCGCCATAATGAGCGCCAGC |
| sequence | CCCGGCGAAAAAGTCACCATCACCTGCAGCGCAAGCAGCAG |
| encoding | CGTCAGCTACATGTACTGGTTCCAACAAAAACCCGGCACCAG |
| light chain | TCCCAAACTCTGGATCTACAGCACCAGCAACCTGGCAAGCGG |
| full-length | CGTGCCCGCCAGATTCAGTGGCAGTGGAAGCGGAACCTCCTA |
| sequence | CAGCCTCACCATCAGCAGAATGGAGGCCGAAGACGCAGCCAC |
| (SEQ ID | CTACTACTGCCAGCAGAGAAGCAGCTACCCATATACCTTCGGA |
| No: 12) | GGCGGGACCAAGCTGGAGATCAAGCGTACGGTGGCTGCACCA |
| | TCTGTCTTCATCTTCCCGCCATCTGATGAGCAGTTGAAATCTG |
| | GAACTGCCTCTGTTGTGTGCCTGCTGAATAACTTCTATCCCAG |
| | AGAGGCCAAAGTACAGTGGAAGGTGGATAACGCCCTCCAATC |
| | GGGTAACTCCCAGGAGAGTGTCACAGAGCAGGACAGCAAGG |
| | ACAGCACCTACAGCCTCAGCAGCACCCTGACGCTGAGCAAAG |
| | CAGACTACGAGAAACACAAAGTCTACGCCTGCGAAGTCACCC |
| | ATCAGGGCCTGAGTTCGCCCGTCACAAAGAGCTTCAACAGGG |
| | GAGAGTGTTGATTCTAGA |

In some embodiments, the antibody or antigen-binding fragment thereof provided herein may contain one or more modifications or substitutions in one or more sequences provided herein, yet retaining specific binding affinity to FGFR2b. Various methods known in the art can be used to achieve this purpose. For example, computer software can be used to virtually simulate the binding of the antibodies to FGFR2b, and identify the amino acid residues on the antibodies which form the binding interface. Such residues may be either avoided in the substitution so as to prevent reduction in binding affinity, or targeted for substitution to provide for a stronger binding.

“Conservatively modified variants” or “conservative substitution” as used herein refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity of the protein. Those skilled in this art would recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally and/or functionally similar amino acids are less likely to disrupt biological activity. Various embodiments of the antibody or antigen-binding fragment thereof according to the present disclosure comprise polypeptide chains with sequences that include up to 0 (no changes), 1, 2, 3, 4, 5, 6, 7, 8, 9, 10, 12, 15, 20, 25, 30, 35, 40, 45, 50 or more conservative amino acid substitutions when compared with the specific amino acid sequences disclosed herein, e.g. SEQ ID NOs: 7, 8, 9 or 10. As used herein, the phrase “up to X” conservative amino acid substitutions includes 0 substitutions and any number of substitutions up to and including X substitutions. Such exemplary substitutions are preferably made in accordance with those set forth in the following table:

TABLE 3
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Functionally conservative variants of the antibody or antigen-binding fragment thereof according to the present disclosure are also contemplated by the present disclosure. “Functionally conservative variants” are those in which one or more amino acid residues in a protein have been changed without altering the overall conformation and function of the polypeptide, including, without limitation, replacement of an amino acid with one having similar properties.

The antibody or antigen-binding fragment thereof provided herein also comprises a constant region capable of inducing effector function. In some embodiments, the constant region comprises one or more modifications which enhances antibody-dependent cellular cytotoxicity (ADCC). In some embodiments, the antibody or antigen-binding fragment thereof is afucosylated.

In some embodiments, the afucosylated antibody can increase effector functions (e.g., ADCC) of an antibody or antigen binding fragment thereof by at least or about 10%, 20%, 30%, 40%, 50%, 60%, 70%, 80%, 90%, 1-fold, 2-fold, 5-fold, 10-fold, 20-fold, 50-fold, or 100-fold, as compared to those of a wild-type antibody or antigen-binding fragment thereof.

As described above, “antibody or antigen-binding fragment thereof” as used herein refers to an intact antibody or an antibody fragment having the antigen-binding portion. Various types of antibodies or antigen-binding fragments are known in the art and can be developed based on the antigen-binding portion of an anti-FGFR2b antibody (e.g., HC18) provided herein.

In some embodiments, the antibody or antigen-binding fragment thereof of the present disclosure is a human antibody, humanized antibody, chimeric antibody, monoclonal antibody, polyclonal antibody, recombinant antibody, diabody, triabody, tetrabody, Fab fragment, F(Fab′)2 fragment, scFv fragment, Fv fragment, Fab′ fragment, or domain antibody.

In some embodiments, the antigen binding fragment provided herein can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

It is also contemplated the antibody or antigen-binding fragment thereof that competes for binding to FGFR2b with the antibody or antigen-binding fragment thereof provided herein (e.g., HC18). In an embodiment, such competitive antibody specifically binds to an epitope that is same as or overlaps with that bound by the antibody or antigen-binding fragment thereof according to the present disclosure. In some embodiments, the antibody or antigen-binding fragment thereof specifically binds to the same or overlapped epitope on FGFR2b recognized by HC18 comprising heavy and light chain variable regions having the amino acid sequences set forth in SEQ ID NOs: 7 and 8, respectively. In some embodiments, the antibody or antigen-binding fragment thereof specifically binds to FGFR2b on a different epitope with FPA144. In some embodiments, the antibody or antigen-binding fragment inhibits the binding interaction between human FGFR2b and an anti-FGFR2b antibody, HC18 by at least 70%, at least 75%, at least 80%, at least 85%, at least 90%, or at least 95%. Those skilled in the art will recognize that it is possible to determine, without undue experimentation, if a given antibody competes for binding to FGFR2b with the antibody of present disclosure.

### Antibody Characteristics

Fibroblast growth factor receptor-2b (FGFR2b) is overexpressed in approximately 2-30% of GC and associated with worse prognosis. Bemarituzumab is an afucosylated humanized monoclonal antibody that specifically inhibits FGFR2b. In the FIGHT trial, bemarituzumab showed beneficial response in GC, but appeared to cause high corneal adverse events. It was hypothesized that FGF10 inhibition may be the mechanism of corneal toxicity by bemarituzumab (Catenacci et al, J Clin Oncol. 2020: 38(21):2418-2426). The present disclosure provides a novel antibody or antigen-binding fragment thereof against FGFR2b with weak inhibition activity for FGF10. Thus, the antibody or antigen-binding fragment thereof may represent a differentiated antibody therapeutics that has a potential to decrease the adverse effects in the eyes.

The antibody or antigen-binding fragment thereof provided herein has at least one of the following properties:
1) specifical binding capabilities to FGFR2b, and indetectable binding affinity to FGFR2c,
2) potent inhibition on the binding of FGF7 to FGFR2b but weak inhibition on the binding of FGF10 to FGFR2b.

In some embodiments, antibody or antigen-binding fragment thereof provided herein can bind to FGFR2b, thereby blocking the interaction of the receptor and their respective ligands; decreasing the phosphorylation of FGFR2; decreasing the phosphorylation of downstream signaling pathways (e.g., MAPK pathway, PI3K/AKT1/MTOR pathway); and/or directly killing the cancer cells by ADCC and/or CDC.

In some embodiments, the antibody or antigen-binding fragment thereof provided herein specifically binds to human FGFR2b with a binding affinity (K_{D}) less than 1×10⁻⁴ M, less than 1×10⁻⁵ M, less than 1×10⁻⁶ M, less than 1×10⁻⁷ M, less than 1×10⁻⁸ M, less than 1×10⁻⁹ M, or less than 1×10⁻¹⁰ M. In some embodiments, the K_{D} is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, the antibody or antigen-binding fragment thereof provided herein specifically binds to human FGFR2b with a comparable binding affinity (K_{D}) with a positive control antibody (e.g., FPA144), for example, as measured by surface plasmon resonance. In some embodiments, the antibody or antigen-binding fragment thereof provided herein has a FGFR2b binding capability that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of FPA144 or FPA144 analog.

In a preferred embodiment, the antibody or antigen-binding fragment thereof provided herein specifically binds to human FGFR2b with a binding affinity (K_{D}) less than 1×10⁻⁷ M, less than 1×10⁻⁸ M, less than 1×10⁻⁹ M, or less than 1×10⁻¹⁰ M.

General techniques for measuring the affinity of an antibody for an antigen include, e.g., ELISA, RIA, and surface plasmon resonance (SPR).

ELISA assay or other common techniques can be used to measure the inhibition of binding of FGF7 and FGF10 to FGFR2b by anti-FGFR2b antibodies. In some embodiments, the antibody or antigen-binding fragment thereof provided herein has a relative inhibition rate of at least about 75%, at least about 80%, at least about 85%, at least about 90%, at least about 95%, or at least about 100% on inhibiting the binding of FGF7 to FGFR2b, as compared with reference antibody (e.g., FPA144). In some embodiments, the antibody or antigen-binding fragment thereof provided herein has a relative inhibition rate of at most about 65%, at most about 60%, at most about 55%, at most about 50%, at most about 45%, or at most about 40% on inhibiting the binding of FGF10 to FGFR2b, as compared with reference antibody (e.g., FPA144).

In some preferred embodiments, the antibody or antigen-binding fragment thereof provided herein has a relative inhibition rate of at least about 85%, at least about 90%, at least about 95%, or at least about 100% on inhibiting the binding of FGF7 to FGFR2b, and has a relative inhibition rate of at most about 55%, at most about 50%, at most about 45%, or at most about 40% on inhibiting the binding of FGF10 to FGFR2b, as compared with reference antibody (e.g FPA144).

In some embodiments, the antibody or antigen-binding fragment thereof provided herein can increase complement dependent cytotoxicity (CDC) by at least 10%, 20%, 30%, 40%, 50%, 60%, 70%, 80%, 90%, 100%, 2 folds, 3 folds, 5 folds, 10 folds, or 20 folds as compared to that of an isotype control antibody.

In some embodiments, the antibody or antigen-binding fragment thereof provided herein can increase antibody-dependent cell-mediated cytotoxicity (ADCC) by at least 10%, 20%, 30%, 40%, 50%, 60%, 70%, 80%, 90%, 100%, 2 folds, 3 folds, 5 folds, 10 folds, or 20 folds as compared to that of an isotype control antibody.

In some embodiments, the antibody or antigen-binding fragment thereof provided herein inhibits FGFR2 phosphorylation and proliferation of cancer cells induced by FGFs.

In some embodiments, the antibody or antigen-binding fragment thereof provided herein decreases the phosphorylation (e.g., FGF7-induced phosphorylation) level of FGFR2 in cells (e.g., FGFR2b-expressing cells) to less than 90%, less than 80%, less than 70%, less than 60%, less than 50%, less than 40%, less than 30%, less than 20%, less than 10%, or less than 5%, as compared to that of a non-specific antibody or an isotype control antibody. In some embodiments, after treatment with the antibody or antigen-binding fragment thereof, the ratio of cells comprising phosphorylated FGFR2 is less than or about 50%, less than or about 60%, less than or about 70%, less than or about 80%, less than or about 90%, less than or about 100%, less than or about 110%, less than or about 120%, less than or about 130%, less than or about 140%, less than or about 150%, less than or about 200%, as compared to that after treatment with FPA144 or FPA144 analog.

In some embodiments, the antibody or antigen-binding fragment thereof provided herein decreases the phosphorylation (e.g., FGF7-induced phosphorylation) level of downstream signaling pathways involved in cancer cell proliferation, survival, and/or apoptosis. In some embodiments, the antibody or antigen-binding fragment thereof provided herein decreases the phosphorylation level of ERK in cells (e.g., FGFR2b-expressing cells) to less than less than 90%, less than 80%, less than 70%, less than 60%, less than 50%, less than 40%, less than 30%, less than 20%, less than 10%, or less than 5% as compared to that of a non-specific antibody, an isotype control antibody. In some embodiments, cells (e.g., FGFR2b-expressing cells) treated with the antibody or antigen-binding fragment thereof have a ratio of phosphorylated ERK1/2 in total ERK of less than or about 90%, less than or about 80%, less than or about 70%, less than or about 60%, less than or about 50%, less than or about 40%, less than or about 30%, less than or about 20%, less than or about 10%, less than or about 5%, as compared to that of cells treated with FPA144 or FPA144 analog.

In some embodiments, the antibody or antigen-binding fragment thereof provided herein has a weaker effect of decreasing the phosphorylation (e.g., FGF10-induced phosphorylation) level of FGFR2 in cells (e.g., FGFR2b-expressing cells) as compared to that of FPA144 or FPA144 analog.

In some embodiments, the antibody or antigen-binding fragment thereof provided herein has a weaker effect of decreasing the phosphorylation (e.g., FGF10-induced phosphorylation) level of downstream signaling pathways involved in cancer cell proliferation, survival, and/or apoptosis as compared to that of FPA144 or FPA144 analog.

In some embodiments, the antibody or antigen-binding fragment thereof provided herein decreases the FGF7-induced proliferation of cells (e.g., FGFR2b-expressing cells) to less than less than 90%, less than 80%, less than 70%, less than 60%, less than 50%, less than 40%, less than 30%, less than 20%, less than 10%, or less than 5% as compared to that of a non-specific antibody, an isotype control antibody.

### Conjugates

The present disclosure provides an antibody-drug conjugate comprising the antibody or antigen-binding fragment thereof linked to one or more conjugate moieties.

In some embodiments, one or more conjugate moieties is a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, one or more conjugate moieties is a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Polynucleotides and Production Methods

The present disclosure also provides a nucleic acid that encode the antibody or antigen-binding fragment thereof provided herein.

The term “nucleic acid” or “polynucleotide” as used herein refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19: 5081 (1991); Ohtsuka et al., J. Biol. Chem. 260: 2605-2608 (1985); and Rossolini et al., Mol. Cell. Probes 8: 91-98 (1994)).

In some embodiments, the nucleic acid encoding the antibody or antigen-binding fragment thereof comprises: a heavy chain encoding nucleic acid having the nucleotide sequence at least 80% (e.g., at least 85%, 90%, 95%, 96%, 97%, 98%, 99% or 100%) identical to SEQ ID NO:11; and/or a light chain encoding nucleic acid having the nucleotide sequence at least 80% (e.g., at least 85%, 90%, 95%, 96%, 97%, 98%, 99% or 100%) identical to SEQ ID NO: 12. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The nucleic acid that encodes the anti-FGFR2b antibody (e.g., including the sequences of HC18 set forth in SEQ ID NO:11 and 12) can be inserted into a vector for further cloning (amplification of the DNA) or for expression, using recombinant techniques known in the art.

The antibody or antigen-binding fragment thereof provided herein can be produced by any method known in the art for synthesis of proteins (e.g., antibodies), especially by chemical synthesis or preferably by recombinant expression techniques.

The recombinant expression of an antibody requires the construction of an expression vector containing a nucleic acid encoding the antibody. Once the nucleic acid encoding the antibody is obtained, a vector for producing the antibody can be produced by recombinant DNA techniques. In the present disclosure, an expression vector is constructed to contain an antibody-coding sequence and appropriate transcription and translation regulatory elements. These methods include, without limitation, in vitro recombinant DNA technologies, synthesis techniques, and in vivo genetic recombination.

The expression vector is transferred to the host cell by conventional techniques, and then the transfected cells were cultured by conventional techniques to produce the antibody or antigen-binding fragment thereof according to the present disclosure.

In an embodiment, a method of producing the antibody or antigen-binding fragment thereof according to the present disclosure comprises culturing the host cell according to the present disclosure under conditions that allow the expression of the antibody or antigen-binding fragment thereof. In a preferred embodiment, the method further comprises recovering and/or purifying the resulted antibody or antigen-binding fragment thereof from the host cell and/or the culture medium.

### Pharmaceutical Composition

The present disclosure provides a pharmaceutical composition comprising the antibody or antigen-binding fragment thereof provided herein, the nucleic acid provided herein, the expression vector provided herein, or the antibody-drug conjugate provided herein, and one or more pharmaceutically acceptable carriers. In a preferred embodiment, the pharmaceutical composition comprises a therapeutically effective amount of the antibody or antigen-binding fragment thereof provided herein, and one or more of additional components, such as a pharmaceutically acceptable carrier, vehicle, or medium. In some embodiments, the pharmaceutical composition comprises a pharmaceutically acceptable carrier can include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

In some embodiments, the compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

The pharmaceutical composition can be administered by any suitable method known to those skilled in the art, such as those parenteral and non-parenteral roots as described above. In a preferred embodiment, the pharmaceutical composition can be administered by intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural or intrasternal injection.

The present disclosure also provides a kit comprising the antibody or antigen-binding fragment thereof provided herein. In a preferred embodiment, the kit according to the present disclosure further comprises an instruction for guiding the use of the antibody or antigen-binding fragment thereof of the present disclosure, such as in treating or preventing a disease associated with the abnormal expression of FGFR2b in a subject, such as a cancer.

In some embodiments, the antibody or antigen-binding fragment thereof provided herein, the nucleic acid provided herein, the expression vector provided herein, or the antibody-drug conjugate provided herein and at least one additional therapeutic agent are administered in a same composition. In some embodiments, the antibody or antigen-binding fragment thereof provided herein, the nucleic acid provided herein, the expression vector provided herein, or the antibody-drug conjugate provided herein and at least one additional therapeutic agent are administered in two different compositions.

In some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

### Therapeutic Method & Use

The present disclosure provides a method of inhibiting or reducing FGF-induced proliferation of tumor cells in a subject, comprising administering a therapeutically effective amount of the antibody or antigen-binding fragment thereof, nucleic acid, expression vector, host cell, antibody-drug conjugate, and/or the pharmaceutical composition provided herein to the subject.

The present disclosure provides a method of inhibiting or reducing FGF-induced FGFR2 phosphorylation in tumor cells of a subject, comprising administering a therapeutically effective amount of the antibody or antigen-binding fragment thereof, nucleic acid, expression vector, host cell, antibody-drug conjugate, and/or the pharmaceutical composition provided herein to the subject.

The present disclosure provides a method of killing tumor cells associated with the abnormal expression of FGFR2b and reducing corneal toxicity in a subject, comprising administering a therapeutically effective amount of the antibody or antigen-binding fragment thereof, nucleic acid, expression vector, host cell, antibody-drug conjugate, and/or the pharmaceutical composition provided herein to the subject.

The present disclosure also provides a method for treating or preventing a disease or condition associated with the abnormal expression of FGFR2b in a subject comprises administering a therapeutically effective amount of the antibody or antigen-binding fragment thereof, nucleic acid, expression vector, host cell, antibody-drug conjugate, and/or the pharmaceutical composition of the present disclosure to the subject.

Further, the antibody or antigen-binding fragment thereof, nucleic acid, expression vector, host cell, antibody-drug conjugate, and/or the pharmaceutical composition provided herein can be used in the manufacture of a medicament and/or kit, such as for treating or preventing a disease or condition associated with the abnormal expression of FGFR2b in a subject.

In some embodiments, the disease or condition is a cancer. In some embodiments, the disease or condition is a cancer characterized in expressing or over-expressing FGFR2b. In some embodiments, the disease or condition is a cancer with FGFR2b mutation, FGFR2b amplification, FGFR2b fusion, FGFR2 translocation, and/or FGFR2 overexpression.

In some embodiments, the cancer includes, without limitation, ovarian cancer, endometrial cancer, breast cancer, lung cancer, bladder cancer, colon cancer, prostate cancer, cervical cancer, colorectal cancer, pancreatic cancer, gastric cancer, esophageal cancer, hepatocellular carcinoma, renal cell carcinoma, head-and-neck cancer, mesothelioma, melanoma, sarcomas, brain tumors, gastroesophageal adenocarcinoma, malignant uterine neoplasm, adenocarcinoma of the gastroesophageal junction, cholangiocarcinoma, intrahepatic cholangiocarcinoma and urothelial caner. In a preferred embodiment, the cancer is gastric cancer. In a preferred embodiment, the cancer is FGFR2 positive gastric cancer. In a preferred embodiment, the cancer is FGFR2-amplified gastric cancer. Patients with cancer can be identified with various methods known in the art.

A therapeutically effective amount of the antibody or antigen-binding fragment thereof, nucleic acid, expression vector, host cell, antibody-drug conjugate, or the pharmaceutical composition provided herein can be administered in one or more administrations. In any of the methods described herein, the antibody or antigen-binding fragment thereof, nucleic acid, expression vector, host cell, antibody-drug conjugate, and/or the pharmaceutical composition provided herein, and, optionally, at least one additional therapeutic agent can be administered to the subject at least once a week (e.g., once a week, twice a week, three times a week, four times a week, once a day, twice a day, or three times a day).

In some embodiments, the subject can be administered the antibody or antigen-binding fragment thereof, nucleic acid, expression vector, host cell, antibody-drug conjugate, and/or the pharmaceutical composition provided herein over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods in the arts for diagnosing or following the effectiveness of treatment (e.g., the observation of at least one symptom of cancer). As described herein, a skilled medical professional can also change the identity and number (e.g., increase or decrease) of the antibody or antigen-binding fragment thereof, nucleic acid, expression vector, host cell, antibody-drug conjugate, and/or the pharmaceutical composition provided herein (and/or one or more additional therapeutic agents) administered to the subject and can also adjust (e.g., increase or decrease) the dosage or frequency of administration of the antibody or antigen-binding fragment thereof, nucleic acid, expression vector, host cell, antibody-drug conjugate, and/or the pharmaceutical composition provided herein (and/or least one other additional therapeutic agents) to the subject based on an assessment of the effectiveness of the treatment (e.g., using any of the methods described herein and known in the art).

In some embodiments, the antibody or antigen-binding fragment thereof, nucleic acid, expression vector, host cell, antibody-drug conjugate, and/or the pharmaceutical composition provided herein is administered with at least one additional therapeutic agent sequentially or simultaneously. In some embodiments, the least one other additional therapeutic agent can be administered to the subject prior to, or after administering the antibody or antigen-binding fragment thereof, nucleic acid, expression vector, host cell, antibody-drug conjugate, and/or the pharmaceutical composition provided herein.

Data obtained from cell culture assays and animal studies can be used in formulating an appropriate dosage of any given agent for use in a subject (e.g., a human). A therapeutically effective amount of the antibody or antigen-binding fragment thereof provided herein will be an amount that treats the disease in a subject, decreases the severity, frequency, and/or duration of one or more symptoms of a disease in a subject (e.g., a human). The effectiveness and dosing of the antibody or antigen-binding fragment thereof provided herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of the antibody or antigen-binding fragment thereof, or antibody-drug conjugate provided herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including the antibody and antigen-binding fragment thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

### EXAMPLES

The present invention is now illustrated by the following examples which should not be construed as limiting.

### Example 1. Preparation of Anti-FGFR2b Antibodies

SJL mice (5-6 week old female) were immunized by intraperitoneal injection at 1 week intervals with FGFR2(beta)IIIb-Fc and anti-CD25/CD40 antibodies (initial anti-CD25 antibody, then dose 100 μg/animal, then anti-CD40 antibody, and finally 2 doses of 50 g/animal), with the antigen suspended in MPL/TDM (Sigma-Aldrich). Three days after the final injection, popliteal lymphoid cells were extracted and fused with Sp2/0-Ag14 mouse myeloma cells at a 1:1 ratio using a Hybrimune Electrofusion System (Cyto Pulse Sciences). Hybridomas were selected by the addition of 2×HAT (Sigma) 24 hours later. Ten days after the fusion, hybridoma culture supernatants were screened for their ability to bind to FGFR2IIIb-his but not to FGFR2IIIc-his using ELISA. Selected mAbs were then screened for their ability to recognize FGFR2IIIb on the CHOK1-FGFR2IIIb transfectants and block the binding of FGF7 to FGFR2IIIb by ELISA. Selected hybridomas were then cloned twice using the limiting dilution technique. Subcloned hybridomas were sequenced and generated by fusing with hFc as chimeric antibodies. A number of anti-FGFR2 chimeric antibodies were obtained from the fusions, including the mAb HC18.

### Example 2. FACS Analysis of Binding Specificities of Anti-FGFR2b Antibodies to Cell Surface Human FGFR2b Protein

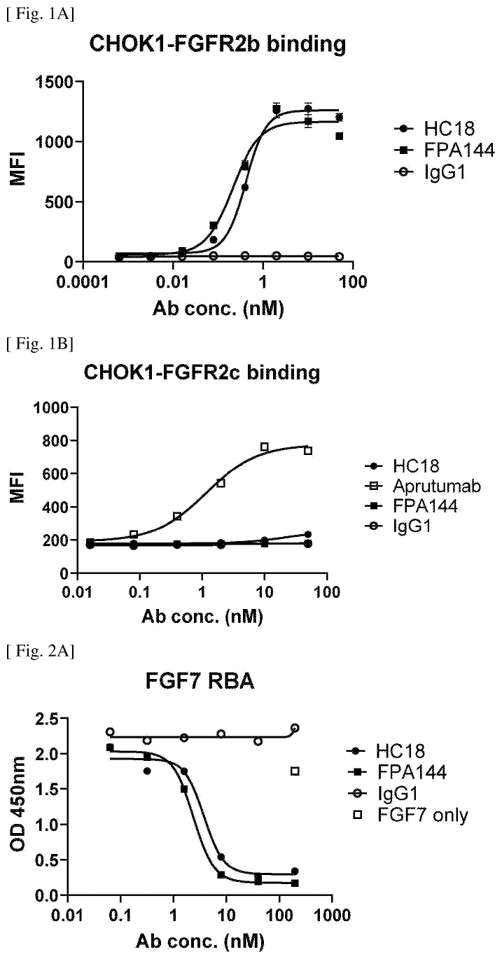

Appropriate cells (1×10⁵) were washed twice in FACS buffer (PBS+2% BSA), resuspended in 100 μl of FACS buffer containing serial diluted (1:5) anti-FGFR2b mAbs, and incubated at 4° C. for 1 hour. Cells were then washed twice in FACS buffer and the bound antibodies were detected by incubation with APC anti-human IgG Fc (Biolegend, Catalog #410712) for 1 hour at 4° C. After washing twice in FACS buffer, cells were analyzed on a Cytoflex (Beckmann Coulter). Aprutumab was also tested as a positive control which can bind to both FGFR2b and FGFR2c. The result is shown in FIG. 1. The antibody of the present disclosure showed comparable binding abilities as compared to Benchmark (FPA144).

### Example 3. Inhibition of Binding of FGF7 and FGF10 to FGFR2b by Anti-FGFR2b Antibodies

Inhibition of binding of FGF7 and FGF10 to FGFR2b by anti-FGFR2b antibodies was measured by ELISA assay. Each step of each assay was performed by room temperature incubation with the appropriate reagent for 1 hour, except the initial plate coating step was done overnight at 4° C. Between each step, plates were washed 3 times in PBS containing 0.05% Tween 20.

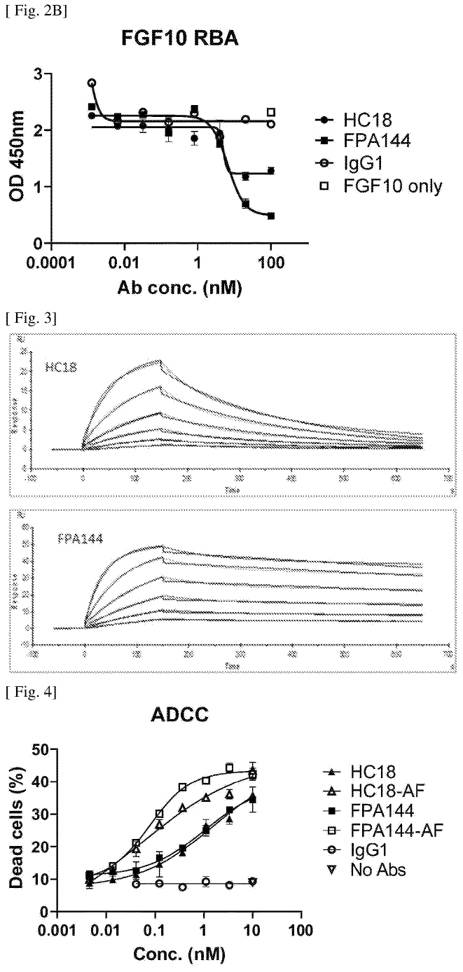

To determine the abilities of the anti-FGFR2b mAbs to block ligand binding to FGFR2b, plates were coated with 1 μg/ml hFGFR2b-Fc (Kactus, Catalog #FGR-HM2BB), and followed by blocking with 2% BSA. The plates were next incubated with human FGF7-his (20 nM, Sino Biological, Catalog #10210-H07E) or biotin-FGF10 (5 nM, Kactus, Catalog #FGF-HE010B) in the presence of serial diluted (1:5) anti-FGFR2b mAbs, and the bound ligands were detected with HRP-anti 6×His tag (Abcam, Catalog #ab1187) or Streptavidin-Protein HRP (Thermo, Catalog #21126) and TMB substrate (Cell Signaling, Catalog #7004P6). The result is shown in FIG. 2 and Table 4. As compared to Benchmark, the antibody of the present disclosure significantly blocked FGF7 binding to FGFR2b but only partially inhibited the binding of FGF10, suggesting that the antibody of the present disclosure may have better safety profile in corneal toxicity.

TABLE 4
Inhibition of binding of FGF7 and FGF10 to FGFR2b

| | FGF7 | | | FGF10 | | |
|---|---|---|---|---|---|---|
| | IC50 | | Inhibition | IC50 | | Inhibition |
| Ab | (nM) | ODmin | (%) | (nM) | ODmin | (%) |
| HC18 | 3.822 | 0.2941 | 94.45% | 4.989 | 1.231 | 54.35% |
| FPA144 | 2.403 | 0.1725 | 100.00% | 6.652 | 0.4962 | 100.00% |

### Example 4. Surface Plasmon Response (SPR) Sensorgrams of Anti-FGFR2b Antibodies Binding to Human FGFR2b Antigen

The binding affinities of anti-FGFR2b mAbs for human FGFR2b (FGFR2b-his) was measured using surface plasmon resonance (Octet, Sartorius). Anti-FGFR2b mAbs were immobilized on a dextran chip using the amine coupling kit and 100 mM ethylenediamine in 100 mM sodium borate, pH 8.0, was used as the blocking reagent. FGFR2b-his proteins diluted in HEPES-buffered saline with 0.05% surfactant P20 running buffer were flowed over the immobilized antibody. The result is shown in FIG. 3 and Table 5.

TABLE 5
Binding affinity of anti-FGFR2b antibody to human FGFR2b.

| Ligand | ka (1/Ms) | kd (1/s) | K_{D} (M) |
|---|---|---|---|
| HC18 | 1.87E+05 | 5.00E−03 | 2.67E−08 |

### Example 5. ADCC for Anti-FGFR2b Antibodies

To generate the afucosylated (AF) mAbs, expression plasmids provided by Client were transformed to E. coli for propagation respectively in appropriate scale. NucleoBond Xtra Maxi Plus EF kit were used for large scale plasmid generation. The constructs containing heavy chain and light chain of each antibody were co-transfected into FUT8-KO CHO cells with PEI. Harvest conditioned medium 9-11 days post transfection. Conditioned medium expressing target Abs were harvested by centrifugation and filtration, then loaded onto Protein A affinity column. Purified Abs were analyzed by SDS-PAGE, SEC-HPLC and endotoxin measurement.

Cytotoxic activity was assessed using FACS analysis. The effect cell human peripheral blood mononuclear cells were obtained from individual human donors (Milestone® Biotechnologies) and cultured with 10 ng/ml hIL-2 (PeproTech, 200-02) overnight. The target Ba/F3 cells expressing full-length human FGFR2b were labeled with Celltrace Far red (Thermo, Catalog #C34564) for 10 minutes at 37° C., washed twice with RPMI 1640 media (Gibco, Catalog #A10491-01) with 10% FBS (Gibco, Catalog #10099-141), and plated at the effector-to-target cell ratios (20:1) in 96-well round-bottom plates. The serial diluted (1:3) anti-FGFR2b mAbs were added to the designated row of the assay plate. After an 4-hour incubation at 37° C., 2 μl Propidium Iodide Staining Solution (BD biosciences, Catalog #556547) was added to each well to dye dead cells at room temperature for 10 minutes. Cells were analyzed on a Cytoflex (Beckmann Coulter) directly. The result is shown in FIG. 4, the antibody of the present disclosure including HC18 and HC18-AF induced strong ADCC response to kill FGFR2b expressing cells and the afucosylated (AF) antibody showed stronger effect.

### Example 6. Inhibition of FGFR2 Phosphorylation Induced by FGF7 and FGF10 of SNU-16 Cells by Anti-FGFR2b Antibodies

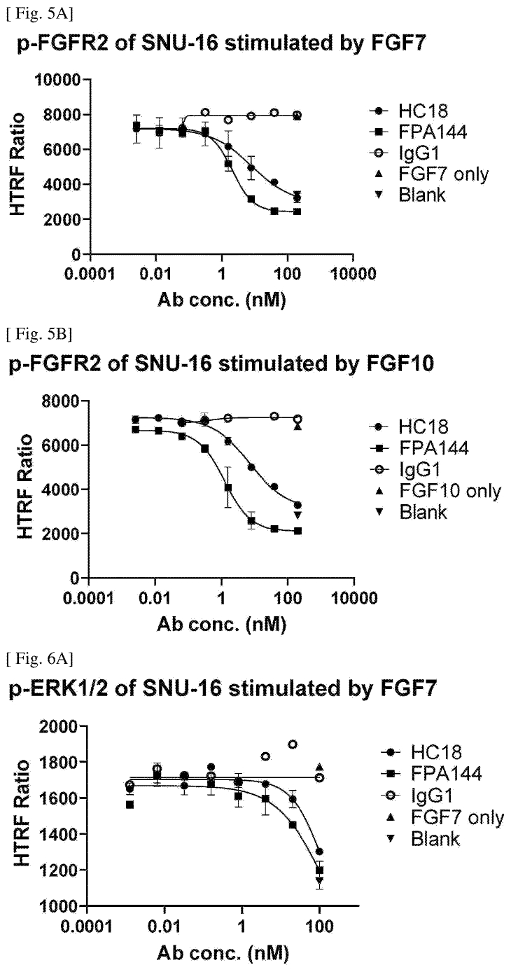

The effects of anti-FGFR2b antibodies on tumor cell FGFR2 phosphorylation in vitro were measured in SNU-16 cells. Approximately 50,000 SNU-16 cells were plated onto a 96-well plate in RPMI 1640 media (Gibco, Catalog #A10491-01) which were incubated at 37° C. with 5% CO₂ for 4 hours. Next, SNU-16 cells were treated with serial diluted (1:10) anti-FGFR2b mAbs for 1 hour. SNU-16 cells were then treated with 30 ng/mL FGF7 (R&D, Catalog #251-KG-01M) or FGF10 (Kactus, Catalog #FGF-HE010B) and 20 μg/mL heparin (Sigma, Catalog #H3149-500KU), and incubated at 37° C. with 5% CO₂ for 5 minutes. To harvest cells for phospho-FGFR2 (Tyr653/654) detection, the 96-well plate was spun down at 300 μg for 3 minutes. The total volume in the well was then slowly aspirated without disrupting the cells. 50 μL of 1× supplemented lysis buffer (Cisbio, Catalog #64FGFR2Y6PEG) was immediately added and incubated for at least 30 minutes at room temperature under shaking, following the manufacturer's protocol for preparation. FGFR2 phosphorylation was read via fluorescence emission at two different wavelengths (665 nm and 620 nm) on a compatible HTRF® reader (EnVision, Perkin Elmer). The experiment was performed in triplicate. The result is shown in FIG. 5, the antibody of the present disclosure significantly inhibited FGFR2 phosphorylation in SNU-16 cells induced by FGF7 and FGF10, especially by FGF7.

### Example 7. Inhibition of ERK1/2 Phosphorylation Induced by FGF7 and FGF10 of SNU-16 Cells by Anti-FGFR2b Antibodies

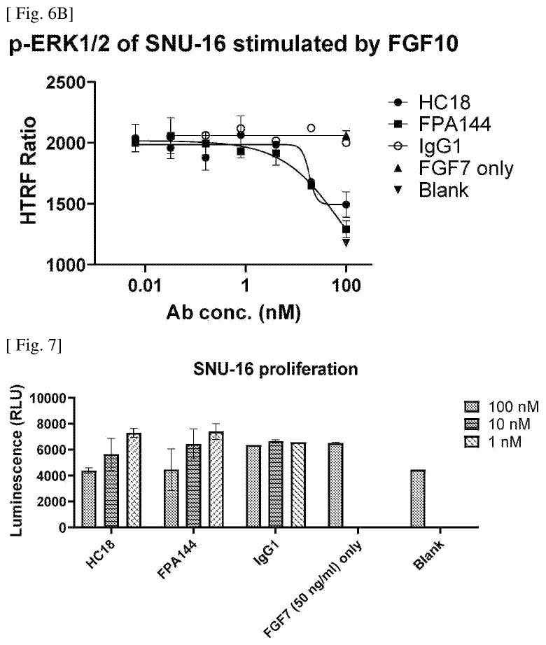

The effects of anti-FGFR2b antibodies on tumor cell ERK1/2 phosphorylation in vitro were measured in SNU-16 cells. Approximately 50,000 SNU-16 cells were plated onto a 96-well plate in RPMI 1640 media (Gibco, Catalog #A10491-01) which were incubated at 37° C. with 5% CO₂ for 4 hours. Next, SNU-16 cells were treated with serial diluted (1:5) anti-FGFR2b mAbs for 1 hour. SNU-16 cells were then treated with 30 ng/mL FGF7 (R&D, Catalog #251-KG-01M) or FGF10 (Kactus, Catalog #FGF-HE010B) and 20 μg/mL heparin (Sigma, Catalog #H3149-500KU), and incubated at 37° C. with 5% CO₂ for 15 minutes. To harvest cells for phospho-ERK (Thr202/Tyr204) detection, the 96-well plate was spun down at 300 g for 3 minutes. The total volume in the well was then slowly aspirated without disrupting the cells. 50 μL of 1× supplemented lysis buffer (Cisbio, Catalog #64ERKPEH) was immediately added and incubated for at least 30 minutes at room temperature under shaking, following the manufacturer's protocol for preparation. ERK1/2 phosphorylation was read via fluorescence emission at two different wavelengths (665 nm and 620 nm) on a compatible HTRF® reader (EnVision, Perkin Elmer). The experiment was performed in triplicate. The result is shown in FIG. 6, the antibody of the present disclosure significantly inhibited ERK1/2 phosphorylation in SNU-16 cells induced by FGF7 and FGF10, especially by FGF7.

### Example 8. Inhibition of the FGF7-Induced Proliferation of SNU-16 Cells by Anti-FGFR2b Antibodies

The effects of anti-FGFR2b antibodies on tumor cell proliferation in vitro were measured in SNU-16 cells. Approximately 10,000 SNU-16 cells were plated onto a 96-well plate in RPMI 1640 media (Gibco, Catalog #A10491-01) to serum starve the cells, which were incubated at 37° C. with 5% CO₂ for 16 hours/overnight. Next, SNU-16 cells were treated with serial diluted (1:10) anti-FGFR2b mAbs for 1 hour. SNU-16 cells were then treated with 50 ng/mL FGF7 (R&D, Catalog #251-KG-01M) and 5 μg/mL heparin (Sigma, Catalog #H3149-500KU), and incubated at 37° C. with 5% CO₂ for 4 days. To harvest cells for the cell viability assay, the 96-well plate was spun down at 300 g for 3 minutes. Half of the total volume in the well (approximately 100 μL) was then slowly aspirated without disrupting the cells. CellTiter-Glo reagent (100 μL) from the CellTiter-Glo Luminescent Cell Viability Assay (Promega, Catalog #G7571) was then added, following the manufacturer's protocol for preparation. Plates were shaken for 15 minutes at room temperature. Plates were protected from light during incubation with the CellTiter-Glo reagent. Cell proliferation was read via luminescence on the SpectraMax M5 (Molecular Devices). The experiment was performed in triplicate. The result is shown in FIG. 7, the antibody of the present disclosure exhibited the same or batter effect as compared to FPA144 on inhibition of SNU-16 proliferation.

### Example 9. Epitope Binning of Anti-FGFR2b mAbs

Epitope binning of anti-FGFR2b antibodies was measured by ELISA assay. Each step of each assay was performed by room temperature incubation with the appropriate reagent for 1 hour, except the initial plate coating step was done overnight at 4° C. Between each step, plates were washed 3 times in PBS containing 0.05% Tween 20.

To determine epitope binning of the anti-FGFR2b mAbs, plates were coated with 1 μg/ml HC18 or BMK FPA144, and followed by blocking with 2% BSA. The plates were next incubated with mixture of 0.1 μg/ml biotin-hFGFR2b-Fc (Kactus, Catalog #FGR-HM2BB) in the presence of serial diluted (1:5) anti-FGFR2b mAbs, and the bound antigen was detected with Streptavidin-Protein HRP (Thermo, Catalog #21126) and TMB substrate (Cell Signaling, Catalog #7004P6). The result is shown in Table 6, indicating that the antibody of the present disclosure has a different epitope bin compared with FPA144.

TABLE 6
Epitope binning of anti-FGFR2b mAbs.

| Competing Ab | FPA144 | HC18 |
|---|---|---|
| FPA144 | 98% | 98% |
| HC18 | 57% | 94% |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Ahn, S., Lee, J., Hong, M., Kim, S. T., Park, S. H., Choi, M. G., Lee, J. H., Sohn, T. S., Bae, J. M., Kim, S., et al. (2016). FGFR2 in gastric cancer: protein overexpression predicts gene amplification and high H-index predicts poor survival. Modern pathology: an official journal of the United States and Canadian Academy of Pathology, Inc 29, 1095-1103.
2. Angal, S., King, D. J., Bodmer, M. W., Turner, A., Lawson, A. D., Roberts, G., Pedley, B., and Adair, J. R. (1993). A single amino acid substitution abolishes the heterogeneity of chimeric mouse/human (IgG4) antibody. Molecular immunology 30, 105-108.
3. Beenken, A., and Mohammadi, M. (2009). The FGF family: biology, pathophysiology and therapy. Nature reviews Drug discovery 8, 235-253.
4. Catenacci, D. V. T., Rasco, D., Lee, J., Rha, S. Y., Lee, K. W., Bang, Y. J., Bendell, J., Enzinger, P., Marina, N., Xiang, H., et al. (2020). Phase I Escalation and Expansion Study of Bemarituzumab (FPA144) in Patients With Advanced Solid Tumors and FGFR2b-Selected Gastroesophageal Adenocarcinoma. Journal of clinical oncology: official journal of the American Society of Clinical Oncology 38, 2418-2426.
5. Grose, R., and Dickson, C. (2005). Fibroblast growth factor signaling in tumorigenesis.

Cytokine & growth factor reviews 16, 179-186.
6. Itoh, N. (2016). FGF10: A multifunctional mesenchymal-epithelial signaling growth factor in development, health, and disease. Cytokine & growth factor reviews 28, 63-69.
7. Katoh, M., and Katoh, M. (2006). FGF signaling network in the gastrointestinal tract (review). International journal of oncology 29, 163-168.
8. Korc, M., and Friesel, R. E. (2009). The role of fibroblast growth factors in tumor growth.

Current cancer drug targets 9, 639-651.
9. Nagatsuma, A. K., Aizawa, M., Kuwata, T., Doi, T., Ohtsu, A., Fujii, H., and Ochiai, A. (2015). Expression profiles of HER2, EGFR, MET and FGFR2 in a large cohort of patients with gastric adenocarcinoma. Gastric cancer: official journal of the International Gastric Cancer Association and the Japanese Gastric Cancer Association 18, 227-238.
10. Ornitz, D. M., Xu, J., Colvin, J. S., McEwen, D. G., MacArthur, C. A., Coulier, F., Gao, G., and Goldfarb, M. (1996). Receptor specificity of the fibroblast growth factor family. J Biol Chem 271, 15292-15297.
11. Turner, N., and Grose, R. (2010). Fibroblast growth factor signalling: from development to cancer. Nature reviews Cancer 10, 116-129.
12. Xiang, H., Chan, A. G., Ahene, A., Bellovin, D. I., Deng, R., Hsu, A. W., Jeffry, U., Palencia, S., Powers, J., Zanghi, J., et al. (2021). Preclinical characterization of bemarituzumab, an anti-FGFR2b antibody for the treatment of cancer. mAbs 13, 1981202.
13. Zhang, X., Ibrahimi, O. A., Olsen, S. K., Umemori, H., Mohammadi, M., and Ornitz, D. M. (2006). Receptor specificity of the fibroblast growth factor family. The complete mammalian FGF family. J Biol Chem 281, 15694-15700.
14. Zheng, W., Ma, M., Du, E., Zhang, Z., Jiang, K., Gu, Q., and Ke, B. (2015). Therapeutic efficacy of fibroblast growth factor 10 in a rabbit model of dry eye. Molecular medicine reports 12, 7344-7350.

1. An antibody or antigen-binding fragment thereof which specifically binds to FGFR2b, comprising: a heavy chain complementarity determining region 1 (CDR1) having the amino acid sequence at least 80% identical to SEQ ID NO: 1, a heavy chain CDR2 having the amino acid sequence at least 80% identical to SEQ ID NO: 2, and a heavy chain CDR3 having the amino acid sequence at least 80% identical to SEQ ID NO: 3.

2. The antibody or antigen-binding fragment thereof of claim 1, having at least one of the following properties:
i) weak inhibition on the binding of FGF10 to FGFR2b;
ii) indetectable binding affinity to FGFR2c.

3. The antibody or antigen-binding fragment thereof of claim 1 or 2, comprising: a light chain CDR1 having the amino acid sequence of SEQ ID NO: 4; a light chain CDR2 having the amino acid sequence of SEQ ID NO: 5; and a light chain CDR3 having the amino acid sequence of SEQ ID NO: 6.

4. The antibody or antigen-binding fragment thereof of claim 3, comprising: a heavy chain CDR1 consisting of the amino acid sequence set forth in SEQ ID NO: 1, a heavy chain CDR2 consisting of the amino acid sequence set forth in SEQ ID NO: 2, a heavy chain CDR3 consisting of the amino acid sequence set forth in SEQ ID NO: 3; and a light chain CDR1 consisting of the amino acid sequence set forth in SEQ ID NO: 4, a light chain CDR2 consisting of the amino acid sequence set forth in SEQ ID NO: 5, and a light chain CDR3 consisting of the amino acid sequence set forth in SEQ ID NO: 6.

5. The antibody or antigen-binding fragment thereof of any of claims 1-4, comprising a heavy chain variable region having the amino acid sequence at least 80% identical to SEQ ID NO: 7; and a light chain variable region having the amino acid sequence at least 80% identical to SEQ ID NO: 8.

6. The antibody or antigen-binding fragment thereof of claim 5, comprising a heavy chain variable region consisting of the amino acid sequence set forth in SEQ ID NO: 7; and a light chain variable region consisting of the amino acid sequence set forth in SEQ ID NO: 8.

7. The antibody or antigen-binding fragment thereof of any of the preceding claims, comprising a heavy chain having the amino acid sequence of SEQ ID NO: 9; and a light chain having the amino acid sequence of SEQ ID NO: 10.

8. The antibody or antigen-binding fragment thereof of any of the preceding claims, comprising an immunoglobulin constant region, optionally a constant region of human immunoglobulin, or optionally a constant region of human IgG, preferably human IgG1.

9. The antibody or antigen-binding fragment thereof of claim 8, wherein the constant region comprises one or more modifications which enhances antibody-dependent cellular cytotoxicity (ADCC).

10. The antibody or antigen-binding fragment thereof of claim 8, which is afucosylated.

11. The antibody or antigen-binding fragment thereof of any of the preceding claims, which is a human antibody, humanized antibody, chimeric antibody, monoclonal antibody, polyclonal antibody, recombinant antibody, diabody, triabody, tetrabody, Fab fragment, F (Fab′)₂ fragment, scFv fragment, Fv fragment, Fab′ fragment, or domain antibody.

12. The antibody or antigen-binding fragment thereof of any of the preceding claims, capable of inhibiting FGFR2 phosphorylation and proliferation of cancer cells induced by FGF.

13. An antibody or antigen-binding fragment thereof which competes for binding to FGFR2b with the antibody or antigen-binding fragment thereof of any of the preceding claims.

14. The antibody or antigen-binding fragment thereof of claim 13, which specifically binds to the same epitope on FGFR2b recognized by an antibody or antigen-binding fragment thereof comprising heavy and light chain variable regions having the amino acid sequences set forth in SEQ ID NOs: 7 and 8, respectively.

15. A nucleic acid encoding the antibody or antigen-binding fragment thereof of any of the preceding claims.

16. The nucleic acid of claim 15, comprising: a heavy chain encoding nucleic acid having the nucleotide sequence of SEQ ID NO: 11; and/or a light chain encoding nucleic acid having the nucleotide sequence of SEQ ID NO: 12.

17. An expression vector comprising the nucleic acid of claim 15 or 16.

18. A host cell comprising the expression vector of claim 17.

19. A pharmaceutical composition comprising:
(a) the antibody or antigen-binding fragment thereof of any one of claims 1-14, the nucleic acid of claim 15 or 16, or the expression vector of claim 17, and
(b) a pharmaceutically acceptable carrier.

20. An antibody-drug conjugate comprising the antibody or antigen-binding fragment thereof of any one of claims 1-14 linked to one or more conjugate moieties.

21. A method of producing the antibody or antigen-binding fragment thereof of any one of claims 1-14, comprising culturing the host cell of claim 18 under conditions that allow the expression of the antibody or antigen-binding fragment thereof.

22. Use of the antibody or antigen-binding fragment thereof of any one of claims 1-14, the nucleic acid of claim 15 or 16, the expression vector of claim 17, the host cell of claim 18, the pharmaceutical composition of claim 19, or the antibody-drug conjugate of claim 20, in the manufacture of a medicament for treating a disease or condition associated with the abnormal expression of FGFR2b in a subject.

23. A method of inhibiting or reducing FGF-induced proliferation of tumor cells in a subject, comprising administering a therapeutically effective amount of the antibody or antigen-binding fragment thereof of any of claims 1-14, the nucleic acid of claim 15 or 16, the expression vector of claim 17, the host cell of claim 18, the pharmaceutical composition of claim 19, or the antibody-drug conjugate of claim 20 to the subject.

24. A method of inhibiting or reducing FGF-induced FGFR2 phosphorylation in a subject, comprising administering a therapeutically effective amount of the antibody or antigen-binding fragment thereof of any of claims 1-14, the nucleic acid of claim 15 or 16, the expression vector of claim 17, the host cell of claim 18, the pharmaceutical composition of claim 19, or the antibody-drug conjugate of claim 20 to the subject.

25. A method of killing tumor cells associated with the abnormal expression of FGFR2b and reducing corneal toxicity in a subject, comprising administering a therapeutically effective amount of the antibody or antigen-binding fragment thereof of any of claims 1-14, the nucleic acid of claim 15 or 16, the expression vector of claim 17, the host cell of claim 18, the pharmaceutical composition of claim 19, or the antibody-drug conjugate of claim 20 to the subject.

26. A method of treating a disease or condition associated with the abnormal expression of FGFR2b in a subject, comprising administering a therapeutically effective amount of the antibody or antigen-binding fragment thereof of any of claims 1-14, the nucleic acid of claim 15 or 16, the expression vector of claim 17, the host cell of claim 18, the pharmaceutical composition of claim 19, or the antibody-drug conjugate of claim 20 to the subject.

27. The use of claim 22 or the method of claim 26, wherein the disease or condition is a cancer, and optionally the cancer is characterized in expressing or over-expressing FGFR2b.

28. The use of claim 22 or the method of claim 26, wherein the disease or condition is one selected from the group consisting of ovarian cancer, endometrial cancer, breast cancer, lung cancer, bladder cancer, colon cancer, prostate cancer, cervical cancer, colorectal cancer, pancreatic cancer, gastric cancer, esophageal cancer, hepatocellular carcinoma, renal cell carcinoma, head-and-neck cancer, mesothelioma, melanoma, sarcomas, brain tumors, gastroesophageal adenocarcinoma, malignant uterine neoplasm, adenocarcinoma of the gastroesophageal junction, cholangiocarcinoma, intrahepatic cholangiocarcinoma and urothelial caner.

29. The use of claim 22 or the method of claim 26, wherein the disease or condition is gastric cancer.

30. The use of claim 22 or the method of claim 26, wherein the disease or condition is FGFR2 positive gastric cancer.

31. The method of any of claims 23-26, wherein the antibody or antigen-binding fragment thereof of any of claims 1-14, the nucleic acid of claim 15 or 16, the expression vector of claim 17, the host cell of claim 18, the pharmaceutical composition of claim 19, or the antibody-drug conjugate of claim 20, is administered with at least one additional therapeutic agent sequentially or simultaneously.

32. A kit comprising the antibody or antigen-binding fragment thereof of any one of claims 1-14.


SEQUENCE LISTING (12 sequences)

SEQ ID NO: 1 - protein, 5 aa, synthetic construct
  REGION 1..5: note = HCDR1 of HC18
SYWMH

SEQ ID NO: 2 - protein, 17 aa, synthetic construct
  REGION 1..17: note = HCDR2 of HC18
SIYPENSDTSYNQKFKG

SEQ ID NO: 3 - protein, 8 aa, synthetic construct
  REGION 1..8: note = HCDR3 of HC18
YHGYDGDY

SEQ ID NO: 4 - protein, 10 aa, synthetic construct
  REGION 1..10: note = LCDR1 of HC18
SASSSVSYMY

SEQ ID NO: 5 - protein, 7 aa, synthetic construct
  REGION 1..7: note = LCDR2 of HC18
STSNLAS

SEQ ID NO: 6 - protein, 9 aa, synthetic construct
  REGION 1..9: note = LCDR3 of HC18
QQRSSYPYT

SEQ ID NO: 7 - protein, 117 aa, synthetic construct
  REGION 1..117: note = VH of HC18
EVQLQQSGTVLARPGASVKMSCKTSGYTFTSYWMHWVKQRPGQGLEWIGSIYPENSDTSYNQKFKGKAKLTAVTSASTAYMDLSSLTNEDSAVYYCSIYHGYDGDYWGQGTTLTVSS

SEQ ID NO: 8 - protein, 106 aa, synthetic construct
  REGION 1..106: note = VL of HC18
QIVLTQSPAIMSASPGEKVTITCSASSSVSYMYWFQQKPGTSPKLWIYSTSNLASGVPARFSGSGSGTSYSLTISRMEAEDAATYYCQQRSSYPYTFGGGTKLEIK

SEQ ID NO: 9 - protein, 447 aa, synthetic construct
  REGION 1..447: note = HC of HC18
EVQLQQSGTVLARPGASVKMSCKTSGYTFTSYWMHWVKQRPGQGLEWIGSIYPENSDTSYNQKFKGKAKLTAVTSASTAYMDLSSLTNEDSAVYYCSIYHGYDGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, synthetic construct
  REGION 1..213: note = LC of HC18
QIVLTQSPAIMSASPGEKVTITCSASSSVSYMYWFQQKPGTSPKLWIYSTSNLASGVPARFSGSGSGTSYSLTISRMEAEDAATYYCQQRSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1351 bp, synthetic construct
  misc_feature 1..1351: note = HC DNA
  source 1..1351: mol_type = other DNA
gaggtgcagctgcagcagagcgggacagtgctggccagaccaggagcaagcgtgaagatgagctgtaagacaagcgggtacacatttacaagctattggatgcactgggtgaagcagagacccggccaaggcctggagtggatcgggagcatctaccccgagaacagcgatacaagctacaaccagaaattcaagggcaaggccaagctgaccgccgtgaccagcgccagcaccgcatacatggacctgagcagcctgaccaacgaggacagcgcagtgtactactgcagcatctaccacggctacgacggagactactggggccaaggcaccactctcaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgattctaga

SEQ ID NO: 12 - DNA, 649 bp, synthetic construct
  misc_feature 1..649: note = LC DNA
  source 1..649: mol_type = other DNA
cagatcgtcctcacccaaagccccgccataatgagcgccagccccggcgaaaaagtcaccatcacctgcagcgcaagcagcagcgtcagctacatgtactggttccaacaaaaacccggcaccagtcccaaactctggatctacagcaccagcaacctggcaagcggcgtgcccgccagattcagtggcagtggaagcggaacctcctacagcctcaccatcagcagaatggaggccgaagacgcagccacctactactgccagcagagaagcagctacccatataccttcggaggcgggaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttgattctaga